(19) United States
(12) Patent Application Publication
(10) Pub. No.: US 2023/0374570 A1
(43) Pub. Date: Nov. 23, 2023

(54) METHOD AND SYSTEM FOR DETECTING FUNGAL GENES AND KIT FOR USE WITH SAME

(71) Applicant: Giner, Inc., Newton, MA (US)

(72) Inventors: Avni A. Argun, Newton, MA (US); Muhit Rana, Southborough, MA (US)

(21) Appl. No.: 18/295,613

(22) Filed: Apr. 4, 2023

Related U.S. Application Data
(60) Provisional application No. 63/327,081, filed on Apr. 4, 2022.

(51) Int. Cl. C12Q 1/6834; C12Q 1/04

(52) U.S. Cl. CPC C12Q 1/6834; C12Q 1/04

(57) ABSTRACT

Method and system for detecting a fungal gene and kit for use with same. According to one embodiment, a nucleotide capture probe is coupled to a surface, such as a well of a 96-well plate, using a biotin-streptavidin interaction. The capture probe is preferably specific for a portion of a target sequence of a fungal gene of interest. Upon capture, additional portions of the target sequence of the fungal gene of interest are tagged with nucleotide labeling probes. An enzyme label, which is preferably a poly-horseradish peroxidase conjugate, is then attached to each of the labeling probes, for example, by a biotin-streptavidin interaction. The enzyme label catalyzes the oxidation of a substrate, such as 3,3′,5,5′-tetramethylbenzidine (TMB) in the presence of hydrogen peroxide. The oxidized substrate may then be detected photonically, by visually detecting a colorimetric change or by absorbance readings, and/or detected electrochemically in the presence of an acid.

### CROSS-REFERENCE TO RELATED APPLICATIONS

The present application claims the benefit under 35 U.S.C. 119(e) of U.S. Provisional Patent Application No. 63/327,081, inventors Avni A. Argun et al., filed Apr. 4, 2022, the disclosure of which is incorporated herein by reference.

### FEDERALLY SPONSORED RESEARCH OR DEVELOPMENT

This invention was made with government support under 75N930020000034 awarded by the Department of Health and Human Services, National Institute of Allergens and Infectious Diseases. The government has certain rights in the invention.

### SEQUENCE LISTING

The instant application contains a Sequence Listing XML which has been submitted electronically in XML file format and is hereby incorporated by reference in its entirety. Said XML copy, created on Apr. 4, 2023, is named 84547A.xml and is 14,757 bytes in size.

### BACKGROUND OF THE INVENTION

The present invention relates generally to techniques for detecting fungal genes and relates more particularly to a novel technique for detecting fungal genes.

Histoplasma capsulatum is a dimorphic fungus that can cause a life-threatening pulmonary infection called histoplasmosis. Histoplasma capsulatum is typically found as a hyaline mold at room temperature; however, when inhaled, the hyaline mold is converted to yeast cells that cause pneumonia. Histoplasma species can be found throughout the world; however, they are endemic to Africa, Central America, and North America. In the United States, they are the most common cause of fungal respiratory infections.

Immunocompromised patients with elevated risk factors, such as bone marrow and organ transplantation, AIDS, leukemia, hematologic malignancy, and chemotherapy, are at 10-15 times higher risk of becoming infected with Histoplasma. People who come into contact daily with soil, such as farmers and construction workers, and who have compromised immune systems have the most significant risk of infection. After a person inhales Histoplasma spores, symptoms usually develop within one to three weeks; however, a diagnosis is not usually determined until six months later. About 50% of the people infected with Histoplasma spores are asymptomatic whereas about 5-10% of cases become severe with long-term lung problems. Despite the prevalence of histoplasmosis, there are only a few drugs approved to treat this infection, and many of these drugs are not only expensive but also toxic with severe side effects; consequently, these drugs are rarely used without a definite diagnosis of histoplasmosis. Therefore, rapid and early diagnosis of histoplasmosis is vital.

Unfortunately, accurate diagnosis of endemic mycoses, like histoplasmosis, has never been straightforward. Despite the prevalence of fungal infections and their threat to human health, there is not a point-of-care (POC) clinical device that is commercially available for early diagnosis of fungal infections like histoplasmosis. Instead, currently available diagnostic techniques, which may take as long as several weeks, often do not provide a definitive diagnosis of fungal infection; moreover, sensitivity and specificity for such techniques are usually low. Chest computerized tomography (CT) scans, biopsy, culture, and microscopy of stained tissue samples can assist with diagnosis; however, techniques involving sampling are highly invasive, and turnaround times can be weeks to months.

The typical diagnostic technique for detecting a fungal infection involves culturing clinical specimens on a suitable media, such as Sabouraud media, followed by a demonstration of the microorganism presence using direct microscopy on stained clinical samples. Unfortunately, however, this diagnostic technique requires a prolonged culturing time and experienced personnel. Other detection methods include antigen detection, serology, histopathology, and cytopathology. Molecular detection methods, like polymerase chain reaction (PCR), offer higher sensitivity and shorter turnaround times; however, such methods require intense sample purification and often yield false positives due to the amplification of non-targets. Additionally, such complex techniques do not readily lend themselves to point-of-care applications. Another technique, namely, fluorescence in situ hybridization (FISH), utilizes fluorescent probes for the detection of DNA sequences; however, FISH requires labeling steps and offers low sensitivity. Finally, in Ostrov et al., “A modular yeast biosensor for low-cost point-of-care pathogen detection,” Sci. Adv., 3: e1603221 (2017), which is incorporated herein by reference, there is disclosed a modular yeast biosensor for POC applications; however, the aforementioned biosensor did not show differential detection for H. capsulatum since the developed sensor also detected Paracoccidioides brasiliensis strains.

In summary, none of the current methods have the required diagnostic sensitivity, rapidity, and clinical utility to detect Histoplasma capsulatum in relevant biological samples in a manner that can be used, if desired, in a point-of-care test setting.

### SUMMARY OF THE INVENTION

It is an object of the present invention to provide a method of detecting a nucleic acid of interest, such as, but not limited to, a fungal gene or a portion of a fungal gene.

It is another object of the present invention to provide a method as described above that addresses at least some of the shortcomings associated with existing methods for detecting nucleic acids of interest.

Therefore, according to one aspect of the invention, there is provided a method of detecting a nucleic acid of interest, the method comprising the steps of (a) providing a surface; (b) securing a capture probe to the surface, wherein the capture probe comprises a capture probe nucleic acid, wherein the capture probe nucleic acid is designed to hybridize with specificity to a first portion of the nucleic acid of interest; (c) then, exposing the capture probe to a sample, wherein, if the sample comprises the nucleic acid of interest, the first portion of the nucleic acid of interest hybridizes to the capture probe nucleic acid of the capture probe; (d) then, adding a first labeling probe, wherein the first labeling probe comprises a first labeling nucleic acid, wherein the first labeling nucleic acid is designed to hybridize with specificity to a second portion of the nucleic acid of interest, wherein, if the nucleic acid of interest is captured, the first labeling probe hybridizes to the second portion of the captured nucleic acid of interest; (e) then, adding an enzyme label, wherein the enzyme label is designed to bind to the first labeling probe, wherein, if the first labeling probe is hybridized to the captured nucleic acid of interest, the enzyme label becomes coupled to the captured nucleic acid of interest; (f) then, adding a substrate whose reaction is catalyzed by the enzyme label, wherein, if the enzyme label is coupled to the captured nucleic acid of interest, the substrate reacts; and (g) then, determining, photonically and/or electrochemically, if any substrate reacts in step (f).

In a more detailed feature of the invention, the nucleic acid of interest may be at least a portion of a fungal gene.

In a more detailed feature of the invention, the fungal gene may be a gene of Histoplasma capsulatum.

In a more detailed feature of the invention, the fungal gene may be selected from the group consisting of the Hcp100 gene, the CBP1 gene, and the M antigen gene.

In a more detailed feature of the invention, the fungal gene may be the Hcp100 gene.

In a more detailed feature of the invention, the surface may be a well of a multiwell plate.

In a more detailed feature of the invention, one of the well and the capture probe may be biotinylated, and the other of the well and the capture probe may be modified with streptavidin.

In a more detailed feature of the invention, the capture probe may be biotinylated, and the well may be coated with streptavidin.

In a more detailed feature of the invention, the multiwell plate may include an electrode.

In a more detailed feature of the invention, the sample may be one of a whole blood sample, a blood plasma sample, a sputum sample, a bronchoalveolar lavage sample, and a sample derived from one or more thereof.

In a more detailed feature of the invention, one of the first labeling probe and the enzyme label may be biotinylated, and the other of the first labeling probe and the enzyme label may be modified with streptavidin.

In a more detailed feature of the invention, the first labeling probe may be biotinylated, and the enzyme label may be modified with streptavidin.

In a more detailed feature of the invention, the method may further comprise, after step (c) and before step (e), adding two or more additional labeling probes, the two or more additional labeling probes comprising a second labeling probe and a third labeling probe, the second labeling probe may comprise a second labeling nucleic acid and the third labeling probe may comprise a third labeling nucleic acid, the second labeling nucleic acid may be designed to hybridize with specificity to a third portion of the nucleic acid of interest and the third labeling nucleic acid may be designed to hybridize with specificity to a fourth portion of the nucleic acid of interest, the enzyme label may also be designed to bind to the second labeling probe and the third labeling probe, and, if the nucleic acid of interest is captured, the second labeling probe may hybridize to the third portion of the captured nucleic acid of interest, and the third labeling probe may hybridize to the fourth portion of the captured nucleic acid of interest.

In a more detailed feature of the invention, the enzyme label may be a streptavidin-modified horseradish peroxidase conjugate.

In a more detailed feature of the invention, the streptavidin-modified horseradish peroxidase conjugate may include five identical horseradish peroxidase homopolymer blocks, and each horseradish peroxidase homopolymer block may comprise 80 horseradish peroxidase monomers.

In a more detailed feature of the invention, the substrate may comprise 3,3′,5,5′-tetramethylbenzidine, and the substrate may be accompanied by hydrogen peroxide.

In a more detailed feature of the invention, the method may further comprise, after step (f) and before step (g), stopping the reaction of the substrate by the addition of an acid.

In a more detailed feature of the invention, photonically determining if any substrate reacts in step (f) may comprise detecting any colorimetric change of the substrate and comparing said colorimetric change, if any, to standards and negative or positive control samples.

In a more detailed feature of the invention, photonically determining if any substrate reacts in step (f) may comprise obtaining an absorbance reading of the substrate and comparing said absorbance reading to standards to determine the amount of any reacted substrate.

In a more detailed feature of the invention, the surface may be a well of a multiwell plate, and the absorbance reading may be taken of any contents of the well while said contents are still in the well.

In a more detailed feature of the invention, electrochemically determining if any substrate reacts in step (f) may comprise performing chronoamperometric analysis of any reacted substrate and comparing said chronoamperometric analysis to standards to determine the amount of any reacted substrate.

In a more detailed feature of the invention, the surface may be a well of a multiwell plate, the well may include an electrode, and the chronoamperometric analysis may be performed on any contents of the well while said contents are still in the well.

In a more detailed feature of the invention, the surface may be a well, and the method may further comprise, after step (c) and before (d), after step (d) and before step (e), and after step (e) and before step (f), removing any non-specific binding from the well.

According to another aspect of the invention, there is provided a system for use in detecting a target nucleic acid, the system comprising (a) a multiwell plate, the multiwell plate comprising a plurality of wells; (b) a plurality of containers, wherein the plurality of containers comprises (i) a first container, the first container comprising a capture probe solution, the capture probe solution comprising a capture probe nucleic acid, wherein the capture probe nucleic acid is designed to bind to a well of the multiwell plate and to hybridize with specificity to a first portion of the target nucleic acid, (ii) a second container, the second container comprising a labeling probe solution, the labeling probe solution comprising a plurality of labeling nucleic acids, each of the plurality of labeling nucleic acids being designed to hybridize with specificity to portions of the target nucleic acid that are different from one another and different from the first portion of the target nucleic acid, (iii) a third container, the third container comprising an enzyme label solution, the enzyme label solution comprising an enzyme label, the enzyme label being designed to bind to the labeling nucleic acids, (iv) a fourth container, the fourth container comprising a substrate solution, the substrate solution comprising a substrate that reacts in the presence of the enzyme label, (c) one or more photonic and/or electrochemical measuring devices, the one or more photonic and/or electrochemical measuring devices being designed to measure the amount of any reacted substrate in the well; (d) a compute device, coupled to the one or more photonic and/or electrochemical measuring devices, for comparing the measured amount of any reacted substrate in the well to standards to determine the amount of the target nucleic acid in the well; and (e) an output device, coupled to the compute device, for displaying the amount of the target nucleic acid in the well.

In a more detailed feature of the invention, at least at least a portion of one well of the multiwell plate may be coated with streptavidin.

In a more detailed feature of the invention, at least one well of the multiwell plate may further comprise an electrode.

In a more detailed feature of the invention, the target nucleic acid may be GAGATCTAGTCGCGGCCAGGTTCACGGAGGACAACGAGTGGTACCGCGCAAAAATA CGGAGAAACGACCGTGAAGCGAAAAAAGCCGACGTCGTTTACATCGACTACGGCAA CTCCGAAA CCGTTCCGTGGAC (SEQ ID NO: 1), the capture probe nucleic acid may be /5Biosg/TG AAC CTG GCC GCG ACT AGA TCT C (SEQ ID NO: 2), and the labeling nucleic acids may be TTG CGC GGT ACC ACT CGT /3Bio/ (SEQ ID NO: 3), CGA TGT AAA CGA CGT CGG CT/3Bio/ (SEQ ID NO: 4), and GTC CAC GGA ACG GTT TCG /3Bio/ (SEQ ID NO: 5).

In a more detailed feature of the invention, the target nucleic acid may be TAT AAA TAT CAG CTC CTT CAC ACT CAG GAA TGG ATG TCT TAC CCT CAA CAT ACA ATC AGC AAG AGA AAA CCC AGC GAA AAT CAC CTC CTC AAT CAA ACA TTC AAA AAA TCT ACG TTC TTT TCC AGA ACA ACC ACT TCG TCA TTC AAA ATG CTT TTC TCC AAG GTT ATC GCT CCT GCT TTC A (SEQ ID NO: 6), the capture probe nucleic acid may be /5Biosg/ GTG TGA AGG AGC TGA TAT TTA TA (SEQ ID NO: 7), and the labeling nucleic acids may be CGC TGG GTT TTC TCT TGC TG /3Bio/ (SEQ ID NO: 8), CGA AGT GGT TGT TCT GGA AA /3Bio/ (SEQ ID NO: 9), and AAA GCA GGA GCG ATA ACC TTG GAG /3Bio/ (SEQ ID NO: 10).

In a more detailed feature of the invention, the target nucleic acid may be GCC ATA AGG ACG TCA CGA AGG GCT TCA TTG CTA CCG TCA CCG ACA GCG CCG ACG GGC TTT CCA TAC GCG TAT GCA TCC GTA ATA ATC CTG AGC GGG CGA CCT CTT GGG TAT TGC GTT GAG GCG CTC GTG AGC AGG CCG CCG ACG ACG ATC ACG GCA TCG AAG ATC GAG CCG TCG GCG CCG GAA TAG GTC ATG TTC ACG C (SEQ ID NO: 11), the capture probe nucleic acid may be /5Biosg/ GAA GCC CTT CGT GAC GTC CTT ATG (SEQ ID NO: 12), and the labeling nucleic acids may be TCA GGA TTA TTA CGG ATG CA /3Bio/ (SEQ ID NO: 13), TTC GAT GCC GTG ATC GTC GT /3Bio/ (SEQ ID NO: 14), and CGT GAA CAT GAC CTA TTC /3Bio/ (SEQ ID NO: 15).

The present invention is also directed at kits that may be usable in the above method and/or system. For example, in one embodiment, where the target nucleic acid is the Hcp100 gene of Histoplasma capsulatum or a portion of said gene, the kit may comprise a combination of a capture probe nucleic acid like /5Biosg/TG AAC CTG GCC GCG ACT AGA TCT C (SEQ ID NO: 2) and a plurality of labeling nucleic acids like TTG CGC GGT ACC ACT CGT /3Bio/(SEQ ID NO: 3), CGA TGT AAA CGA CGT CGG CT/3Bio/ (SEQ ID NO: 4), and GTC CAC GGA ACG GTT TCG /3Bio/ (SEQ ID NO: 5). In another embodiment, where the target nucleic acid is the CBP1 gene of Histoplasma capsulatum or a portion of said gene, the kit may comprise a combination of a capture probe nucleic acid like /5Biosg/ GTG TGA AGG AGC TGA TAT TTA TA (SEQ ID NO: 7) and a plurality of labeling nucleic acids like CGC TGG GTT TTC TCT TGC TG /3Bio/ (SEQ ID NO: 8), CGA AGT GGT TGT TCT GGA AA /3Bio/ (SEQ ID NO: 9), and AAA GCA GGA GCG ATA ACC TTG GAG /3Bio/ (SEQ ID NO: 10). In still another embodiment, where the target nucleic acid is the M antigen gene of Histoplasma capsulatum or a portion of said gene, the kit may comprise a combination of a capture probe nucleic acid like /5Biosg/ GAA GCC CTT CGT GAC GTC CTT ATG (SEQ ID NO: 12), and a plurality of labeling nucleic acids like TCA GGA TTA TTA CGG ATG CA /3Bio/ (SEQ ID NO: 13), TTC GAT GCC GTG ATC GTC GT /3Bio/ (SEQ ID NO: 14), and CGT GAA CAT GAC CTA TTC /3Bio/ (SEQ ID NO: 15).

Additional objects, as well as aspects, features, and advantages, of the present invention will be set forth in part in the description which follows, and in part will be obvious from the description or may be learned by practice of the invention. In the description, reference is made to the accompanying drawings which form a part thereof and in which is shown by way of illustration various embodiments for practicing the invention. The embodiments will be described in sufficient detail to enable those skilled in the art to practice the invention, and it is to be understood that other embodiments may be utilized and that structural changes may be made without departing from the scope of the invention. The following detailed description is, therefore, not to be taken in a limiting sense, and the scope of the present invention is best defined by the appended claims.

### BRIEF DESCRIPTION OF THE DRAWINGS

The accompanying drawings, which are hereby incorporated into and constitute a part of this specification, illustrate various embodiments of the invention and, together with the description, serve to explain the principles of the invention. The drawings are not necessarily drawing to scale, and certain components may have undersized and/or oversized dimensions for purposes of explication. In the drawings wherein like reference numeral represents like parts:

FIG. 1 is a flowchart illustrating one embodiment of a method for detecting a fungal gene in accordance with the present invention;

FIG. 2 is a simplified schematic view of one embodiment of a system that may be used to perform the method of FIG. 1;

FIG. 3 is a schematic representation of one embodiment of the method of FIG. 1;

FIG. 4A is a graph illustrating the absence test and assay performance evaluation of the assay in phosphate-buffered saline (PBS) buffer, as discussed in Example 3, Part (G);

FIGS. 4B and 4C are graphs illustrating sensitivity testing of the Hcp100 gene in PBS using spectroscopic measurement at 450 nm and electrochemical amperometric peak analysis, respectively, as discussed in Example 3, Part (H);

FIG. 5A is a graph illustrating that signals obtained from an absorbance measurement at 450 nm of TMB (3,3′,5,5′-tetramethylbenzidine) are strong enough to distinguish from background signal, as discussed in Example 3, Part (I);

FIGS. 5B and 5C are graphs illustrating absorbance after employing statistical background correction absorbance measurement in % from CBP1 and M antigen genes, respectively, as discussed in Example 3, Part (I);

FIGS. 6A and 6B are graphs illustrating spectroscopic analysis and electrochemical analysis, respectively, of whole blood samples for the CBP1 gene, as discussed in Example 3, Part (J);

FIG. 6C is a graph illustrating a standard curve with R2=0.9966 value from electrochemical amperometry peak analysis, as discussed in Example 3, Part (J);

FIGS. 6D and 6E are graphs illustrating spectroscopic and electrochemical sensitivity of testing, respectively, for the CBP1 gene in whole blood samples, as discussed in Example 3, Part (J);

FIGS. 7A through 7G are graphs illustrating electrochemical chronoamperometry measurements of CBP1 in blood samples at concentrations of 0 M, 100 aM, 1 fM, 10 fM, 10 pM, 100 pM, and 1 nM, respectively, as discussed in Example 3, Part (J);

FIG. 8 is a graph illustrating the representative sensitivity in log scale obtained from electrochemical chronoamperometry measurements of CBP1 in blood samples at concentrations of 0 M, 100 aM, 1 fM, 10 fM, 1 nM and 10 nM, as discussed in Example 3, Part (J);

FIGS. 9A and 9B are graphs illustrating specificity testing of the CBP1 gene in whole blood samples and buffer samples using absorbance and chronoamperometry readings, respectively, as discussed in Example 3, Part (K);

FIGS. 9C and 9D are graphs illustrating specificity testing of the M antigen gene in whole blood samples and buffer samples using absorbance and chronoamperometry readings, respectively, as discussed in Example 3, Part (K);

FIGS. 10A and 10B are graphs illustrating specificity testing of the Hcp100 gene target using absorbance and chronoamperometry readings, respectively, as discussed in Example 3, Parts (K) and (Q);

FIG. 11 is a graph illustrating positive control testing of the CBP1 gene in PBS and BAL (bronchoalveolar lavage) samples using chronoamperometry readings, as discussed in Example 3, Part (L);

FIGS. 12A and 12B are graphs illustrating CBP1 gene detection in blood and BAL samples, respectively, using absorbance readings, as discussed in Example 3, Parts (L) and (R);

FIG. 13 is a graph, using absorbance readings, to illustrate the difference between nucleic acid hybridization for single-stranded DNA (ssDNA) and double-stranded DNA (dsDNA), as discussed in Example 3, Part (M);

FIG. 14 is a graph illustrating fluorogenic characterization of the Hcp100 target (ssDNA) and its sequence specific probe to form dsDNA, as discussed in Example 3, Part (N);

FIG. 15 is a graph of absorbance readings, illustrating the specificity in buffer of the Hcp100 gene target even in the presence of a non-target, KPC gene, as discussed in Example 3, Part (O); and

FIG. 16 is a graph of optical density readings, showing the reproducibility of the assay after different time intervals, as discussed in Example 3, Part (P).

### DETAILED DESCRIPTION OF THE INVENTION

As noted above, the rapid and accurate diagnosis of endemic fungal diseases is an urgent medical need. The public health impact of fungal infections is increasingly becoming more widespread as fungal infections have high morbidity and mortality, especially in immune-compromised patients. Unfortunately, current medical diagnostics are insufficient to meet adequate clinical requirements. By contrast, the present invention provides a low-cost and robust technique or assay for rapidly and accurately identifying the presence of specific fungal genes. More specifically, according to one embodiment, the present invention may be used to provide an early and definitive detection of one or more genes that are present in the fungus Histoplasma capsulatum; consequently, the present invention may be used to provide a rapid diagnosis of the fungal infection known as histoplasmosis.

In at least one embodiment, the technique of the present invention may involve the nucleic acid hybridization of a target nucleic acid that is indicative or specific to a fungus of interest to a nucleotide capture probe. In at least one embodiment, prior to the aforementioned nucleic acid hybridization, the nucleotide capture probe may be secured to a surface, such as a well of a multiwell plate; thus, the nucleic acid hybridization of the target nucleic acid to the nucleotide capture probe may effectively secure the target nucleic acid to the aforementioned surface. In at least one embodiment, the securing of the aforementioned nucleotide capture probe to the aforementioned surface may involve a biotin-streptavidin interaction. For example, the surface may be coated with streptavidin, and the nucleotide capture probe may be biotinylated.

Additionally, in at least one embodiment, the technique of the present invention may involve the nucleic acid hybridization of one or more nucleotide labeling (or amplification) probes to the thus-captured target nucleic acid. In at least one embodiment, each of the aforementioned one or more nucleotide labeling probes may be coupled to a label. In at least one embodiment, the aforementioned label may be an enzyme label, such as horseradish peroxidase. More specifically, each of the aforementioned one or more nucleotide labeling probes may be biotinylated, and the enzyme label may comprise a streptavidin poly-horseradish peroxidase conjugate that may bind to a nucleotide labeling probe via a biotin-streptavidin interaction. More specifically, after coupling the enzyme label to the target nucleic acid via the nucleotide labeling probes (and washing away any non-specific binding), the enzyme label may be exposed to an enzyme substrate. For example, where the enzyme label is a streptavidin poly-horseradish peroxidase conjugate, the enzyme substrate may be 3,3′,5,5′-tetramethylbenzidine (TMB). Horseradish peroxidase catalyzes the oxidation of TMB; consequently, after exposing the bound horseradish peroxidase to a substrate solution containing TMB, the presence of oxidized TMB may be used to indicate the presence of the target nucleic acid. In at least one embodiment, the presence of oxidized TMB may be detected photonically and/or electrochemically.

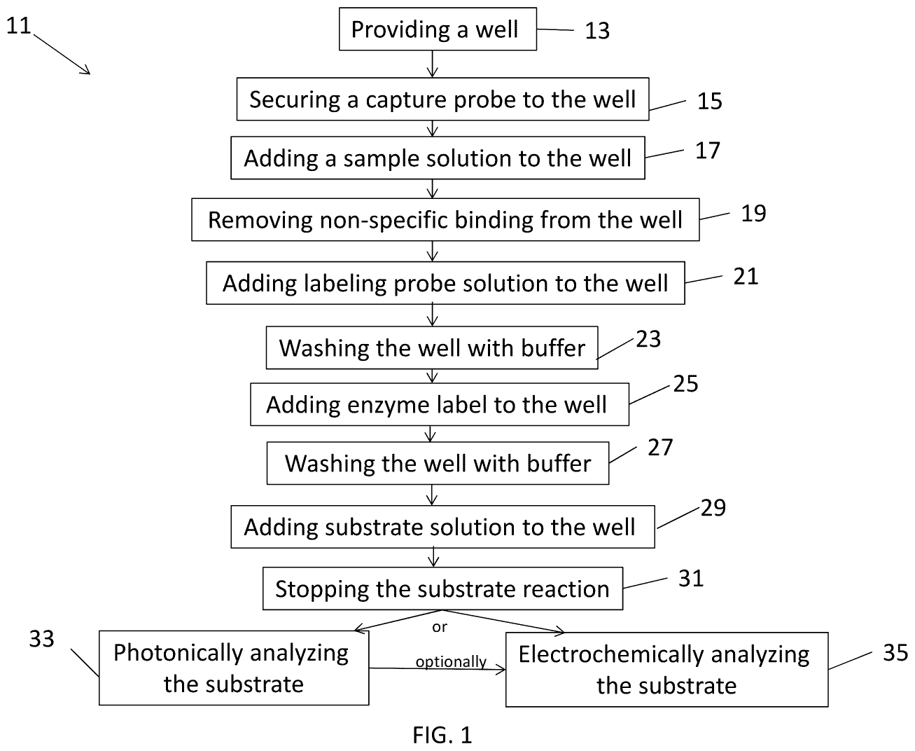

Referring now to FIG. 1, there is shown a flowchart illustrating one embodiment of a method for detecting a fungal gene according to the present invention, the method being represented generally by reference numeral 11. Details of method 11 that are discussed elsewhere in this application or that are not critical to an understanding of the invention may be omitted from FIG. 1 and/or from the accompanying description herein or may be shown in FIG. 1 and/or described herein in a simplified manner.

Method 11 may comprise a step 13 of providing a well or cavity. According to one embodiment, the well or cavity may be, for example, a well of a conventional streptavidin-coated multiwell (e.g., 96-well) plate.

Method 11 may further comprise, preferably after step 13, a step 15 of securing a capture probe to the well or cavity. As will be discussed further below, the capture probe may comprise a relatively short nucleotide sequence, which may be, for example, a single-stranded nucleic acid (e.g., DNA, RNA, etc.) of approximately 20-25 nucleotides, designed to hybridize with high specificity to a first portion of a target nucleic acid of a fungal gene of interest. According to one embodiment, the fungal gene of interest may be a gene that is common to Histoplasma capsulatum and other fungi or may be a gene that is specific to Histoplasma capsulatum. The target nucleic acid may be a single-stranded or double-stranded nucleic acid (e.g., DNA, RNA, etc.) and may correspond to a portion of the fungal gene of interest or may correspond to the entirety of the fungal gene of interest. Alternatively, the target nucleic acid may be a single-stranded or double-stranded nucleic acid that is derived from a portion of the fungal gene of interest or the entirety of the fungal gene of interest. Examples of fungal genes of interest that may be used to detect Histoplasma capsulatum may include the Hcp100 gene (GenBank: AJ005963.1), the CBP1 gene (Azimova et al., “Cpb1, a fungal virulence factor under positive selection, forms an effector complex that drives macrophage lysis,” PLoS Pathog., 18(6): e1010417 (2022) which is incorporated herein by reference), and the M antigen gene (GenBank: AF026268.2; Guedes et al., “PCR assay for identification of Histoplasma capsulatum based on the nucleotide sequence of the M antigen,” J Clin Microbiol., 41(2): 535-9 (2003), which is incorporated herein by reference).

The 5′-end of the capture probe may be biotinylated so that the capture probe may bind to the well of a streptavidin-coated multiwell plate via a biotin-streptavidin interaction. According to one embodiment, a quantity of the biotinylated capture probe in a buffer solution may be added to a streptavidin-coated well and may be allowed to bind, via a biotin-streptavidin interaction, to the streptavidin-coated well for a period of time.

Although, in the present embodiment, the securing of the capture probe to the well is preferably effected using a streptavidin-coated well and a biotinylated capture probe, it is to be understood that the securing of the capture probe to the well need not be effected in this manner. Rather, for example, the well may be coated with biotin, and the 5′-end of the capture probe may be modified with streptavidin. Moreover, the securing of the capture probe to the well may be effected by alternative mechanisms that do not employ a biotin-streptavidin interaction. Such alternative mechanisms may involve covalent bonding mechanisms or non-covalent bonding mechanisms.

Method 11 may further comprise, preferably after step 15, a step 17 of adding a sample solution to the well. The sample solution may be, for example, a biological specimen obtained from one or more subjects, which biological specimen may be a sample of whole blood, blood plasma, sputum, bronchoalveolar lavage fluid, or any other suitable biological material. Alternatively, the sample solution may consist of or comprise a solvent or carrier spiked with whole blood, sputum, bronchoalveolar lavage fluid, or other suitable biological material, or a component of one or more of the foregoing, or the sample solution may consist of or comprise a biological specimen or component therefrom that has been treated in some fashion to facilitate the detection of the target nucleic acid therein.

The sample solution is preferably added to the well in such a manner that the contents of the sample solution are exposed to the capture probe for a period of time. In this manner, if the sample solution contains the target nucleic acid of the gene of interest, the target nucleic acid will bind to the capture probe via hybridization of complementary nucleotide sequences.

Method 11 may further comprise, preferably after step 17, a step 19 of removing any non-specific binding from the well. Step 19 may be effected by washing the well one or more times, preferably three times, with a buffer solution, such as 1× phosphate buffered saline-Tween (PBS-T) buffer. In addition, following the aforementioned washing, the well may be treated with a biotin solution for a period of time, such as 10 minutes.

Method 11 may further comprise, preferably after step 19, a step 21 of adding a quantity of a labeling probe solution to the well. In the present embodiment, the labeling probe solution may comprise one or more nucleotide labeling probes, preferably three different nucleotide labeling probes, in a buffer solution. As will be discussed further below, the one or more nucleotide labeling probes may be relatively short nucleotide sequences, which may be, for example, single-stranded nucleic acids (e.g., DNA, RNA, etc.) of approximately 20-25 nucleotides, designed to hybridize with high specificity to the target nucleic acid of the fungal gene of interest at different portions of the target nucleic acid than the portion of the target nucleic acid to which the capture probe is hybridized and at different portions of the target nucleic acid than the portions to which the other nucleotide labeling probes are hybridized. In the present embodiment, the three nucleotide labeling probes are designed so that, when hybridized to the target nucleic acid, each nucleotide labeling probe is spaced apart from its neighboring labeling probes and/or capture probe by a small number of nucleotides (e.g., approximately 1-6 nucleotides). For reasons that will become apparent below, the 3′-end of each nucleotide labeling probe may be biotinylated. The labeling probe solution may be added to the well, and hybridization may be allowed to occur for a period of time.

Method 11 may further comprise, preferably after step 21, a step 23 of washing the well with buffer solution to remove any non-specific binding of the nucleotide labeling probes.

Method 11 may further comprise, preferably after step 23, a step 25 of adding an enzyme label to the well. In the present embodiment, the enzyme label may be a streptavidin-modified horseradish peroxidase conjugate. An example of a suitable streptavidin-modified horseradish peroxidase conjugate may be Streptavidin-PolyHRP80 conjugate, which includes five identical horseradish peroxidase homopolymer blocks, each block containing 80 horseradish peroxidase monomers and being covalently coupled to multiple streptavidin molecules. The enzyme label may be added to the well as part of an enzyme label solution and may be exposed to the one or more hybridized nucleotide labeling probes in such a way that, due to biotin-streptavidin interactions, enzyme label binds to each of the hybridized nucleotide labeling probes.

As can be appreciated, because, in the present embodiment, multiple nucleotide labeling probes are used to hybridize to a single target nucleic acid, and each nucleotide labeling probe binds an enzyme label, and each enzyme label includes multiple horseradish peroxidase enzymes, the amount of enzyme label that can be complexed with the target nucleic acid may be greatly amplified. As a result, the reaction that is catalyzed by the enzyme label may be more easily detected. Moreover, because the nucleotide labeling probes of the present invention are highly specific for the target nucleic acid, the present invention also reduces false positive results that may otherwise occur if a nucleic acid other than the targeted nucleic acid were to bind to the capture probe.

Also, although, in the present embodiment, the securing or coupling of the enzyme label to the one or more hybridized nucleotide labeling probes is preferably effected using a biotinylated nucleotide labeling probe and a streptavidin-modified horseradish peroxidase conjugate, it is to be understood that the securing of the enzyme label to the one or more hybridized nucleotide labeling probes need not be effected in this manner. Rather, for example, the enzyme label may be biotinylated, and the 3′-ends of the one or more nucleotide labeling probes may be modified with streptavidin. Moreover, the securing of the enzyme label to the one or more hybridized nucleotide labeling probes may be effected by alternative mechanisms that do not employ a biotin-streptavidin interaction. Such alternative mechanisms may involve covalent bonding mechanisms or non-covalent bonding mechanisms. Furthermore, it is to be understood that enzyme labels other than horseradish peroxidase may be used.

Method 11 may further comprise, preferably after step 25, a step 27 of washing the well to remove any non-specific binding of the enzyme label.

Method 11 may further comprise, preferably after step 27, a step 29 of adding a substrate solution to the well. Where, as in the present embodiment, the enzyme label comprises hydrogen peroxidase, the substrate solution may comprise hydrogen peroxide and a substrate of 3,3′,5,5′-tetramethylbenzidine (TMB). Hydrogen peroxidase catalyzes the oxidation of TMB in the presence of hydrogen peroxide. Consequently, if the target nucleic acid of the fungal gene of interest has been captured by the capture probe, hydrogen peroxidase will be coupled to the target nucleic acid via the nucleotide labeling probes; thus, upon addition of the substrate solution to the well, TMB will be oxidized. By contrast, if the target nucleic acid of the fungal gene of interest has not been captured by the capture probe, hydrogen peroxidase will not be present in the well; thus, upon addition of the substrate solution to the well, TMB will not be oxidized.

Method 11 may further comprise, preferably after step 29, a step 31 of stopping the substrate reaction, which, in the present embodiment, is the oxidation of TMB. In the present embodiment, the oxidation of TMB may be stopped after a period of time (e.g., approximately 10 minutes) by the addition of a quantity of an acidic solution to the well. The acidic solution may be, for example, a sulfuric acid solution (e.g., a 0.5M sulfuric acid solution).

Method 11 may further comprise a step 33, which may occur after step 31, of photonically analyzing the contents of the well, particularly any oxidized substrate that may be present in the well. In the present embodiment, the foregoing photonic analysis may comprise, for example, visually noting the color of the contents of the well and comparing the noted color to appropriate standards for known quantities of the oxidized substrate. For example, if oxidized TMB is present and has been exposed to an acid (as in step 31), the contents of the well will typically exhibit a dark yellow or orange color whereas, if oxidized TMB is not present, the contents of the well will typically be clear (i.e., exhibit no color) or a very pale yellow. Additionally and/or alternatively, the photonic analysis of the contents of the well may comprise measuring the light absorbance or optical density of the well contents at one or more wavelengths. For example, the absorbance of the well contents may be measured at approximately 450 nm and then compared to appropriate standards obtained for known amounts of oxidized substrate. The determined amount of oxidized substrate can, in turn, be used to determine the amount of the gene of interest in the sample and, thus, the amount of the fungus of interest present in the sample.

As can be appreciated, measuring absorbance may provide more sensitive quantitative information than using a colorimetric change.

It should be understood that, although, in the present embodiment, step 33 occurs after step 31, step 33 need not occur after step 31 and may occur, for example, before step 31 or in the absence of a step corresponding to step 31. here, for example, step 31 does not precede step 33 or step 31 does not occur, the photonic analysis of step 33 may still comprise visually noting the color of the contents of the well and comparing the noted color to appropriate standards obtained for known contents. For example, if oxidized TMB is present but has not been exposed to an acid (as in step 31), the contents of the well will typically exhibit a dark blue color whereas, if oxidized TMB is not present, the contents of the well will typically be clear (i.e., exhibit no color) or a very pale blue. Additionally and/or alternatively, the photonic analysis of the contents of the well may comprise measuring the light absorbance or optical density of the well contents at approximately 650 nm and then comparing to appropriate standards obtained for known contents.

Method 11 may further comprise, preferably after step 31, a step 35 of electrochemically analyzing the contents of the well, particularly any oxidized substrate that may be present in the well. In the present embodiment, the electrochemical analysis may comprise, for example, chronoamperometric analysis of the oxidized substrate, wherein such analysis is focused on the cathodic current that is produced when any oxidized TMB present is reduced under acidic conditions. Current peaks that are observed during this reduction step are proportional to the amount of TMB in solution and, thus, are proportional to the targeted nucleic acid of the fungal gene of interest. Therefore, by measuring the current peaks and then comparing the measured amounts to appropriate standards for the oxidized substrate, one may determine the amount of the fungal gene of interest that is present, and, correspondingly, the amount of fungus that is present. Step 35 may be performed by transferring the unbound contents of the well to an electrode, such as a screen-printed carbon electrode, followed by chronoamperometric measurements. The aforementioned electrode may be, for example, a screen-printed carbon electrode formed in the well of another multiwell plate or may be a well of a carbon-coated or carbon-based multiwell plate. Alternatively, the aforementioned electrode may be present within the original well using, for example, a streptavidin-coated carbon electrode, wherein the carbon electrode is screen-printed onto the well, in which case the chronoamperometric measurements may be conducted without transferring the contents of the well to another location.

As can be appreciated, the electrochemical analysis of step 35 may be conducted instead of, or in addition to, the photonic analysis of step 33 or may not be conducted at all. Electrochemical analysis may be preferred to photonic analysis (whether colorimetric or light absorbance) in terms of its inherent sensitivity and may be especially advantageous where the sample being tested is turbid or otherwise does not lend itself to analysis through color change or light absorbance. In any event, because chronoamperometric analysis is preferably conducted under acidic conditions, if step 35 follows step 33 and if step 33 is conducted in the absence of step 31, acid may need to be added to the well contents before step 35 may be conducted.

Also, as can be appreciated, where a multiwell (e.g., 96-well) plate is used to perform method 11, one may use multiple wells of the same plate to detect the presence of the same fungal gene of interest or may use different wells of the same plate to detect the presence of different fungal genes of interest. For example, the same capture probe and the same set of nucleotide labeling probes may be used in all of the wells of the same plate to detect the presence of the same fungal gene of interest. Alternatively, a first capture probe and a first set of nucleotide labeling probes may be used in a first set of one or more wells of the plate to detect a first fungal gene of interest, and a second capture probe and a second set of nucleotide labeling probes may be used in a second set of one or more wells of the same plate to detect a second fungal gene of interest. Whether the same plate is used to detect a single fungal gene of interest or a plurality of fungal genes of interest, different wells may be used to test samples derived from the same source or from different sources.

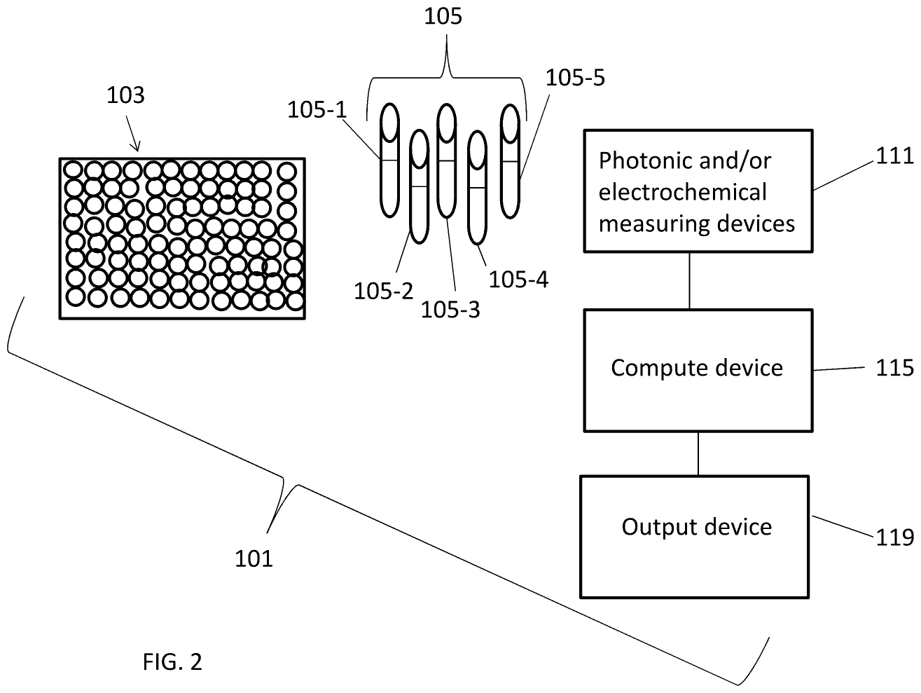

Referring now to FIG. 2, there is shown a simplified schematic view of one embodiment of a system that may be used to perform the method of FIG. 1, the system being represented generally by reference numeral 101. Details of system 101 that are discussed elsewhere in this application or that are not critical to an understanding of the invention may be omitted from FIG. 2 and/or from the accompanying description herein or may be shown in FIG. 2 and/or described herein in a simplified manner.

System 101 may comprise a multiwell plate 103. In the present embodiment, multiwell plate 103 may be a 96-well plate and, more specifically, may be a conventional streptavidin-coated 96-well plate or a streptavidin-coated carbon 96-well plate.

System 101 may further comprise a plurality of containers 105 containing various reagents or materials for use in detecting a fungal gene of interest. For example, the plurality of containers 105 may comprise a first container 105-1 containing a capture probe solution (which may be used, for example, in step 15 of method 11), a second container 105-2 containing a labeling probe solution (which may be used, for example, in step 21 of method 11), a third container 105-3 containing an enzyme label solution (which may be used, for example, in step 25 of method 11), a fourth container 105-4 containing a substrate solution (which may be used, for example, in step 29 of method 11), and a fifth container 105-5 containing an acidic stop solution (which may be used, for example, in step 31 of method 11). Although not shown, the plurality of containers 105 may also comprise additional containers containing, for example, washing solutions or the like.

System 101 may further comprise one or more photonic and/or electrochemical measuring devices 111 for use in detecting any oxidized substrate in the wells of the multiwell plate 103. To detect and/or to quantify photonically the amount of oxidized substrate that may be present in a well, the one or more photonic and/or electrochemical measuring devices 111 may comprise, for example, a spectrophotometer. More specifically, the spectrophotometer may be designed to take one or more absorbance readings at a wavelength that is indicative of oxidized TMB (e.g., about 450 nm after the addition of an acid stop solution; about 650 nm prior to the addition of an acid stop solution).

Alternatively, to detect and/or to quantify electrochemically the amount of oxidized substrate that may be present in a well, the one or more photonic and/or electrochemical measuring devices 111 may comprise, for example, an electroelectrochemical 96-well plate reader and a potentiostat. The reader and potentiostat may be used in the manner described above to perform chronoamperometric analysis of the oxidized substrate. (As can be appreciated, if electrochemical detection is to be used, the multiwell plate 103 must include an electrode or the unbound contents of the well must be transferred to a suitable electrode.) The aforementioned 96-well plate reader may be a Metrohm CONNECTOR96X reader (Metrohm USA, Inc., Riverview, FL), which can simultaneously read up to eight different electrochemical signals from eight different wells of an electrode well plate. This instrument is also able to dynamically switch between different columns of the well plate to scan all 96 wells without input from the user. The aforementioned potentiostat may be, for example, a Metrohm μSTAT 4000 potentiostat (Metrohm USA, Inc., Riverview, FL), which has four potentiostats and, therefore, is able to simultaneously apply a potential to up to four different electrodes in the 96-well plate. The foregoing potentiostat is preferably connected to a Metrohm SYNCONN96X sync connector (Metrohm USA, Inc., Riverview, FL), which sends a signal to the 96-well plate reader to switch columns when the current scan has concluded.

System 101 may further comprise a compute device 115. Compute device 115 may be coupled, either wirelessly or via one or more wires, to the one or more photonic and/or electrochemical measuring devices 111. Compute device 115 may be designed to receive the measurements from the one or more photonic and/or electrochemical measuring devices 111 and to compare the measurements to standards so as to quantify the amount of fungal gene and/or fungus present in the sample tested.

System 101 may further comprise an output device 119. Output device 119 may be coupled, either wirelessly or via one or more wires, to compute device 115 and may comprise a monitor, printer, or other suitable device for outputting and/or displaying the results of the quantification made by compute device 115.

The following examples are given for illustrative purposes only and are not meant to be a limitation on the invention described herein or on the claims appended hereto.

### EXAMPLES

### Example 1 —Target and Probe Sequences

The Hcp100 consensus sequences among five Histoplasma genomes (see Voorhies et al., “Chromosome-Level Genome Assembly of a Human Fungal Pathogen Reveals Synteny among Geographically Distinct Species,” mBio, 13(1): e0257421 (February 2022), which is incorporated herein by reference) were used to design a capture probe, three labeling probes, and a target sequence to specifically detect Histoplasma strains. The Hcp100 gene is specific to Histoplasma strains whereas the ITS2 and 18S rRNA regions are not. Therefore, as a first step, a highly conserved region of the Hcp100 gene was selected as a target to determine the presence of Histoplasma DNA. In addition, analogous sequences for the CBP1 and M antigen regions were also designed to demonstrate preclinical utility. The designed sequences for the Hcp100 gene are shown below in TABLE 1.

TABLE 1
DNA sequences designed for detection of the Hcp100 gene

| Name | Sequence (5′→3′) | Length |
|---|---|---|
| Target (Hcp100) | GAGATCTAGTCGCGGCCAGGTTCACGGAG | 133 |
| | GACAACGAGTGGTACCGCGCAAAAATACGG | |
| | AGAAACGACCGTGAAGCGAAAAAAGCCGAC | |
| | GTCGTTTACATCGACTACGGCAACTCCGAAA | |
| | CCGTTCCGTGGAC (SEQ ID NO: 1) | |
| C.probe_Hcp100 | /5Biosg/TG AAC CTG GCC GCG ACT AGA TCT C | 24 |
| (capture probe) | (SEQ ID NO: 2) | |
| P1_Hcp100 | TTG CGC GGT ACC ACT CGT/3Bio/ | 18 |
| (first labeling | (SEQ ID NO: 3) | |
| probe) | | |
| P2_Hcp100 | CGA TGT AAA CGA CGT CGG CT/3Bio/ | 20 |
| (second labeling | (SEQ ID NO: 4) | |
| probe) | | |
| P3_Hcp100 | GTC CAC GGA ACG GTT TCG/3Bio/ | 18 |
| (third labeling | (SEQ ID NO: 5) | |
| probe) | | |

Besides the ITS2 rRNA gene, alternative conserved target gene sequences of Histoplasma are a 192-bp region of GAPDH gene tested with 797 clinical samples (see Babady et al., “Detection of Blastomyces dermatitidis and Histoplasma capsulatum from Culture Isolates and Clinical Specimens by Use of Real-Time PCR, J. Clin. Microbiol., 49(9): 3204-8 (2011); and Rudramurthy et al., “Molecular Genetics and Genomics of Fungal Infections,” in Mehmet Turgut, Sundaram Challa and Ali Akhaddar (eds.), Fungal Infections of the Central Nervous System: Pathogens, Diagnosis, and Management (Springer International Publishing: Cham) (2019), both of which are incorporated herein by reference), the rRNA 18S gene (see da Silva et al., “Fluorescent in situ hybridization of pre-incubated blood culture material for the rapid diagnosis of histoplasmosis, Med Mycol, 53: 160-4 (2015); and Persaud et al., “Comparison of Urine Antigen Assays for the Diagnosis of Histoplasma captsulatum Infection,” The Journal of Applied Laboratory Medicine, 4(3): 370-82 (2019), both of which are incorporated herein by reference), the Hcp100 gene locus (see Scheel et al., “Development of a Loop-Mediated Isothermal Amplification Method for Detection of Histoplasma capsulatum DNA in Clinical Samples,” Journal of Clinical Microbiology, 52(2): 483-8 (2014), which is incorporated herein by reference) or encoding N-acetyl α-linked acidic dipeptidase (NAALADase) (see Dufresne et al., “Diagnosis of Systemic Fungal Diseases,” in Amar Safdar (ed.), Principles and Practice of Transplant Infectious Diseases (Springer New York: New York, NY) (2019), which is incorporated herein by reference), all of which were identified in either human patient blood, serum, FFPE or BAL fluid with consideration of diagnosis of histoplasmosis per European Organization for Research and Treatment of Cancer (EORTC) criteria (see Muraosa et al., “Detection of Histoplasma capsulatum from clinical specimens by cycling probe-based real-time PCR and nested real-time PCR, Med Mycol, 54(4): 433-8 (2016), which is incorporated herein by reference).

Even though the ITS2 region is not as specific as the Hcp100 gene, it is more abundant in the fungi genome. (The Hcp100 gene is found as only one copy per genome.) Therefore, this region can be targeted as a control. Probes that target the ITS2 region of Histoplasma were designed as shown below in TABLES 2 and 3. Two candidate genes (CBP1 and M antigen) were down-selected for sequencing and commercial synthesis of probes and target due to their high specificity to Histoplasma. As a proof-of-concept, the Hcp100 gene was continuously utilized for testing the assay sensitivity, specificity and exogenous Hcp100 gene detection in commercial whole blood. After initial optimization of Hcp100 gene detection, CBP1 and M antigen gene targets, their capture probes, and labeling probes sequences were designed.

TABLE 2
DNA sequences designed for detection of the CBP1 gene

| Name | Sequence (5′→3′) | Length |
|---|---|---|
| Target (CBP1) | TAT AAA TAT CAG CTC CTT CAC ACT CAG GAA | 181 |
| | TGG ATG TCT TAC CCT CAA CAT ACA ATC AGC | |
| | AAG AGA AAA CCC AGC GAA AAT CAC CTC CTC | |
| | AAT CAA ACA TTC AAA AAA TCT ACG TTC TTT | |
| | CTT TTC TCC AAG GTT ATC GCT CCT GCT TTC A | |
| | (SEQ ID NO: 6) | |
| C.probe_CBP1 | /5Biosg/GTG TGA AGG AGC TGA TAT TTA TA | 23 |
| (capture probe) | (SEQ ID NO: 7) | |
| P1_CBP1 | TCC AGA ACA ACC ACT TCG TCA TTC AAA ATG | |
| (first labeling | CGC TGG GTT TTC TCT TGC TG/3Bio/ | 20 |
| probe) | (SEQ ID NO: 8) | |
| P2_CBP1 | CGA AGT GGT TGT TCT GGA AA/3Bio/ | |
| (second labeling | (SEQ ID NO: 9) | 20 |
| probe) | | |
| P3_CBP1 | AAA GCA GGA GCG ATA ACC TTG GAG/3Bio/ | 24 |
| (third labeling | (SEQ ID NO: 10) | |
| probe) | | |

TABLE 3
DNA sequences designed for detection of the M antigen gene

| Name | Sequence (5′→3′) | Length |
|---|---|---|
| Target | GCC ATA AGG ACG TCA CGA AGG GCT TCA TTG | 199 |
| (M antigen) | CTA CCG TCA CCG ACA GCG CCG ACG GGC TTT | |
| | CCA TAC GCG TAT GCA TCC GTA ATA ATC CTG | |
| | AGC GGG CGA CCT CTT GGG TAT TGC GTT GAG | |
| | GCG CTC GTG AGC AGG CCG CCG ACG ACG ATC | |
| | ACG GCA TCG AAG ATC GAG CCG TCG GCG CCG | |
| | GAA TAG GTC ATG TTC ACG C (SEQ ID NO: 11) | |
| C.probe_M | /5Biosg/GAA GCC CTT CGT GAC GTC CTT ATG | 24 |
| antigen | (SEQ ID NO: 12) | |
| (capture probe) | | |
| P1_M antigen | TCA GGA TTA TTA CGG ATG CA/3Bio/ | 20 |
| (first labeling | (SEQ ID NO: 13) | |
| probe) | | |
| P2_M antigen | TTC GAT GCC GTG ATC GTC GT/3Bio/ | |
| (second labeling | (SEQ ID NO: 14) | 20 |
| probe) | | |
| P3_M antigen | CGT GAA CAT GAC CTA TTC/3Bio/ | 18 |
| (third labeling | (SEQ ID NO: 15) | |
| probe) | | |

### Example 2—Optimization of Histoplasma DNA Isolation Using Commercially Available Kits

Efforts were undertaken to optimize the DNA isolation protocols for Histoplasma. To this end, two commercially available DNA extraction kits from Qiagen N.V. (Venlo, The Netherlands) were tested for efficiency in isolating DNA from untreated, fixed, heat-killed, and ethanol-killed Histoplasma yeast cells: QIAGEN DNeasy Blood and Tissue Kit (CAT#69504, hereinafter “Blood and Tissue Kit”) and QIAGEN DNeasy PowerLyzer Microbial Kit (CAT#12255-50, hereinafter “PowerLyzer Kit”). Portions of the protocols were adjusted to facilitate performance in a wide range of laboratory environments, as well as to account for difficulty in lysing Histoplasma yeast cells.

First, Histoplasma capsulatum strain G217B yeast cells were harvested in two different ways, denoted as “Sample set 1.” A portion of the cells was left untreated, and the remaining was fixed with formaldehyde. Pellets from these two groups were frozen at −20° C. for storage. Both the Blood and Tissue Kit and the PowerLyzer Kit were used to isolate DNA from these pellets.

The standard protocol for the Blood and Tissue Kit was modified according to portions of the supplementary protocol developed for yeast cells, optimized for difficult-to-lyse starting material. This involved re-suspending the cell pellet in a higher concentration of proteinase K than the standard, followed by a 15-minute incubation at 56° C. and additional vortexing. The protocol for the PowerLyzer Kit indicates two options for homogenization instruments: the PowerLyzer 24 and a standard multi-tube vortex. The vortex was used, as it is present in many laboratories, unlike the PowerLyzer 24. The fixed cells were first processed in the same way as the untreated cells. Then, in another test, the fixed cells were incubated at 70° C. for 1 hour in an attempt to reverse the crosslinking induced by the fixation procedure and to enable more efficient cell lysis.

Under all of the conditions tested, the DNA isolation was largely inefficient in the fixed cells. While the Blood and Tissue Kit inefficiently extracted the DNA from the untreated cells, as well as the fixed cells, the PowerLyzer Kit showed promise as this protocol led to efficient DNA isolation from the untreated samples. For this reason, efforts were undertaken to move forward with optimizing the PowerLyzer Kit only. In TABLES 4 through 6 below, the concentrations and other quantitative measurements are listed for the isolated DNA from each sample.

### Blood and Tissue Kit

Sample set 1
Eluted in 100 uL
Initial steps modified:
   1. Add 180 uL Buffer ATL and 20 uL proteinase K to pellet. Vortex to combine.
   2. Incubate at 56° C. for 15 minutes, vortexing again every 5 minutes.
3. Vortex for 15 seconds.
   4. Add 200 uL Buffer AL. Vortex to combine.
   5. Add 200 uL ethanol (96-100%). Vortex to combine.
   6. Continue protocol from step 4 (loading sample into spin column).

TABLE 4
Concentrations and other quantitative
measurements for isolated DNA

| | Concentration | Absorbance at | Absorbance at |
|---|---|---|---|
| | [ng/uL] | 260 nm/280 nm | 260 nm/230 nm |
| Untreated 1 | 15.3 | 2.29 | 0.43 |
| Untreated 2 | 11.1 | 2.31 | 0.31 |
| Fixed 1 | 3.5 | 1.55 | 0.23 |
| Fixed 2 | 5.5 | 2.45 | 0.19 |

The ratio of absorbance at 260 nm and 280 nm is used to assess the purity of DNA and RNA. A ratio of ˜1.8 is generally accepted as “pure” for DNA; a ratio of ˜2.0 is generally accepted as “pure” for RNA. If the ratio is appreciably lower in either case, it may indicate the presence of protein, phenol or other contaminants that absorb strongly at or near 280 nm. The ratio of absorbance at 260 nm and 230 nm is used as a secondary measure of nucleic acid purity. The 260/230 values for “pure” nucleic acid are often higher than the respective 260/280 values. Expected 260/230 values are commonly in the range of 2.0-2.2. If the ratio is appreciably lower than expected, it may indicate the presence of contaminants which absorb at 230 nm.

### PowerLyzer Kit

Sample set 1
Eluted in 50 uL
Exact protocol, using 10-minute vortex to homogenize (step 4)

TABLE 5
Concentrations and other quantitative
measurements for isolated DNA

| | Concentration | Absorbance at | Absorbance at |
|---|---|---|---|
| | [ng/uL] | 260 nm/280 nm | 260 nm/230 nm |
| Untreated 1 | 196.4 | 1.97 | 2.38 |
| Untreated 2 | 204.3 | 1.98 | 2.36 |
| Fixed 1 | 1.5 | 1.56 | 0.17 |
| Fixed 2 | 2.3 | 0.85 | 0.54 |

### PowerLyzer Kit

Sample set 1
Eluted in 50 uL
Initial steps modified:
   1. For fixed samples, re-suspend pellet in 300 uL PowerBead Solution (Qiagen, N.V., Venlo, The Netherlands). Add 50 uL Solution SL. Vortex to combine. Incubate at 70° C. for 1 hour.
   2. For untreated samples, re-suspend pellet in 300 uL PowerBead Solution. Add 50 uL Solution SL. Vortex to combine. Incubate at 70° C. for 10 minutes.
   3. Transfer samples to PowerBead tubes. Vortex at top speed for 10 minutes to homogenize.
   4. Continue protocol from step 5 (centrifugation).

TABLE 6
Concentrations and other quantitative
measurements for isolated DNA

| | Concentration | Absorbance at | Absorbance at |
|---|---|---|---|
| | [ng/uL] | 260 nm/280 nm | 260 nm/230 nm |
| Untreated 1 | 144.1 | 1.90 | 2.17 |
| Untreated 2 | 157.0 | 1.92 | 2.34 |
| Fixed 1 | 3.7 | 2.88 | 1.09 |
| Fixed 2 | 4.6 | 2.61 | 1.73 |

Because the PowerLyzer Kit did not perform well with fixed cells, two other methods of killing the Histoplasma yeast cells were tested: heat and ethanol. Histoplasma capsulatum strain G217B yeast cells were harvested in three different ways, denoted as “Sample set 2” below. A portion of the cells was left untreated, a portion was incubated at 95° C. for 20 minutes (heat-killed), and a portion was exposed to a 75% ethanol solution (ethanol-killed). Pellets from these two groups were frozen at −20° C. for storage. The PowerLyzer Kit was used to isolate DNA from these pellets.

DNA isolation was inefficient in the heat-killed cells; however, the PowerLyzer Kit protocol was effective in extracting DNA from the ethanol-killed cells. TABLE 7 below lists the concentrations and other quantitative measurements for the isolated DNA from each sample.

### PowerLyzer Kit

Sample set 2
Eluted in 50 uL
Exact protocol, using 10-minute vortex to homogenize (step 4).

TABLE 7
Concentrations and other quantitative
measurements for isolated DNA

| | Concentration | Absorbance at | Absorbance at |
|---|---|---|---|
| | [ng/uL] | 260 nm/280 nm | 260 nm/230 nm |
| Untreated 1 | 23.6 | 2.16 | 1.66 |
| Untreated 2 | 21.9 | 2.15 | 1.17 |
| Heat-Killed 1 | 4.9 | 2.05 | 0.68 |
| Heat-Killed 2 | 6.1 | 2.56 | 0.70 |
| Ethanol-Killed 1 | 16.0 | 2.15 | 0.93 |
| Ethanol-Killed 2 | 18.9 | 2.17 | 1.59 |

### Example 3—Assay for Detection of Fungal Gene

(A) Materials: All of the oligonucleotides of TABLES 1, 2 and 3 (i.e., the custom capture probes, the three labeling probes, and the target sequences for the Hcp100, CBP1, and M antigen gene regions of H. capsulatum) were designed by one or more of the present inventors and obtained from Integrated DNA Technologies, Inc. (Coralville, IA). Streptavidin Poly-HRP80 conjugate (Catalog #65R-S120) was purchased from Fitzgerald Industries International (Acton, MA). Streptavidin-coated 96-well plates and BLOCKER bovine serum albumin (BSA) (10%) in phosphate-buffered saline (PBS) (Catalog #37525) were purchased from Thermo Fischer Scientific Inc. (Waltham, MA). 3,3′,5,5′-tetramethylbenzidine (TMB) substrate solution was bought from Surmodics IVD Inc. (Eden Prairie, MN). 20× PBS-T (PBS-TWEEN 20) buffer was obtained from Thermo Fisher Scientific (Waltham, MA) whereas 10× PBS buffer (pH 7.4), TE (Tris-EDTA) buffer (pH 8.0), and diethyl pyrocarbonate (DEPC)-treated water were procured from Invitrogen, a brand of Thermo Fischer Scientific. Biotin (Catalog # B4501), H₂SO₄, MgCl₂, and all other reagents were purchased from Sigma-Aldrich (St. Louis, MO). Invitrogen's Ultrapure DNase-free, RNase-free DEPC treated water (catalog #4387937) was used in all studies. All commercial blood and bronchoalveolar lavage (BAL) (de-identified pooled) fluids were purchased from BioIVT (Westbury, NY). Screen-printed carbon electrodes (Metrohm Dropsens DRP-110) were purchased from Metrohm USA Inc. (Riverview, FL). A USB-powered potentiostat (Model number: EmStat3+, Potential range ±3 V or ±4 V, and Current ranges 1 nA to 10 mA or 100 mA) was obtained from PalmSens BV (Houten, The Netherlands).

(B) Experimental stock preparation: DNA oligonucleotides were re-suspended in TE buffer and stored at −20° C. as DNA stocks. DNA probe immobilization and target hybridization buffers were prepared with 1× PBS buffer, including 20 mM MgCl₂. In addition, 1× Phosphate Buffered Saline-Tween (PBS-T) buffer and 1× PBS buffer were prepared as washing solutions from the buffer stock solutions. The poly-HRP80-Streptavidin conjugate was diluted in 1× PBS buffer, including 1% BSA at required dilution rates. In addition, 40 μM of freshly prepared biotin solution was prepared in nuclease free DEPC-treated water. H₂SO₄ stock solution was diluted with deionized (DI) water to obtain 0.5 M H₂SO₄ solution. DEPC-treated water was used for all other solution preparation throughout the experiments.

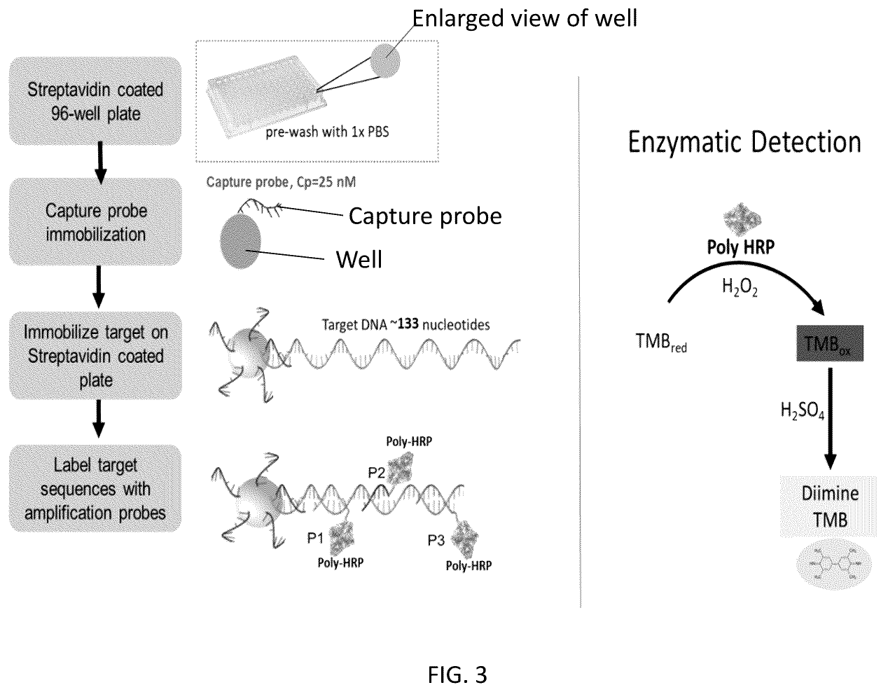

(C) Assay Development: As shown in FIG. 3, an enzymatic sensing system was developed to detect fungal genomic DNA utilizing a single-stranded capture probe (Cp) and three labeling oligonucleotide probes (P1, P2, and P3). The capture probe was designed to hybridize with a nucleic acid region on single-stranded target DNA. The capture probe was modified with biotin at its 5′ end to attach to the streptavidin-coated 96-well plate surface. After 25 nM of capture probe was immobilized on the plate surface, a sample solution containing single-stranded target DNA molecules was added to the well. Synthetic target DNA molecules were designed as a 133-bp oligonucleotide for Hcp100, a 181-bp oligonucleotide for CBP1, and a 93-bp oligonucleotide for M antigen.

After hybridization, the wells were washed three times with 1× PBS-T buffer, and each well was treated with 40 μM of freshly prepared biotin solution for 10 min at room temperature (RT) to minimize non-specific bindings. 100 uL of the labeling probe mixture, which was previously prepared in one tube to be 25 nM of each the P1, P2, and P3 labeling probes, was added to each of the wells to hybridize along the length of the target DNA. After washing the wells with buffer solution, 30× Streptavidin Poly-HRP80 conjugate (prepared with 10×of 10% Blocker BSA solution with PBS) was introduced to the wells to attach to the labeling probes by biotin-streptavidin interaction. After the washing step, 100 μL of stock TMB was added to the wells. Poly-HRP catalyzes the oxidation of TMB in the presence of hydrogen peroxide. After 10 minutes, 50 μL of 0.5M H₂SO₄ was added to the wells to stop the reaction. Each well was analyzed by UV/Vis spectroscopy (at 450 nm) and chronoamperometry. The oxidized form of TMB can be measured by chronoamperometry once converted to the diimine form in acidic conditions.

(D) Sensitivity measurements: Spectrophotometric and electrochemical measurements were performed using various concentrations of target DNAs designed separately for the M antigen, Hcp100, and CBP1 gene regions. Ranging concentrations of target DNA (i.e., 10 nM down to 100 aM for each of the M antigen and CBP1 genes; 1 nM down to 61 fM for the Hcp100 gene) were added to the wells to hybridize with 25 nM of capture probes. Control experiments were performed in the absence of the target gene while keeping every other parameter unchanged. For all electrochemical measurements, 70 uL of TMB solution was added onto the electrode surface, followed by amperometric measurements. An operating potential of 125 mV was applied to decay the transient currents to a steady-state value. The time required for this was determined to be 30 seconds for this study. Sharply increasing current implies that TMB reduction has been achieved.

(E) Specificity measurements: The specificity of the assay was evaluated for each target sequence tested. A complex cocktail mixture of non-target genes was prepared to show that the assay can detect only the targeted gene sequence in vitro PBS buffer, as well as in exogenously enriched commercial human whole blood and BAL samples.

(F) Detection in Blood and BAL Samples: The unprocessed (no anticoagulant/filtration, storage condition at −20° C. or colder) human whole blood and BAL samples were utilized to demonstrate the assay preclinical utility. Prior to testing, 5 μL of blood and/or BAL samples were diluted with 95 μL of reaction buffer to a final volume of 100 μL (20× dilution). The specimens (Blood and BAL samples) were spiked with 1 nM of the target gene, CBP1. Hcp100 and M antigen genes were tested in exogenously enriched commercial human whole blood. Both exogenously enriched (spiked) and unspiked samples were tested, followed by the remaining enzymatic steps as described above.

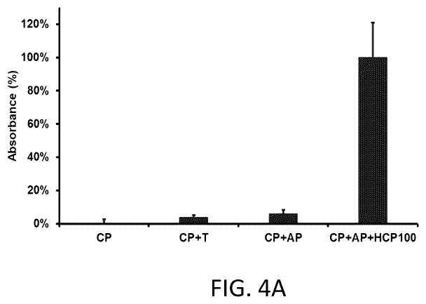

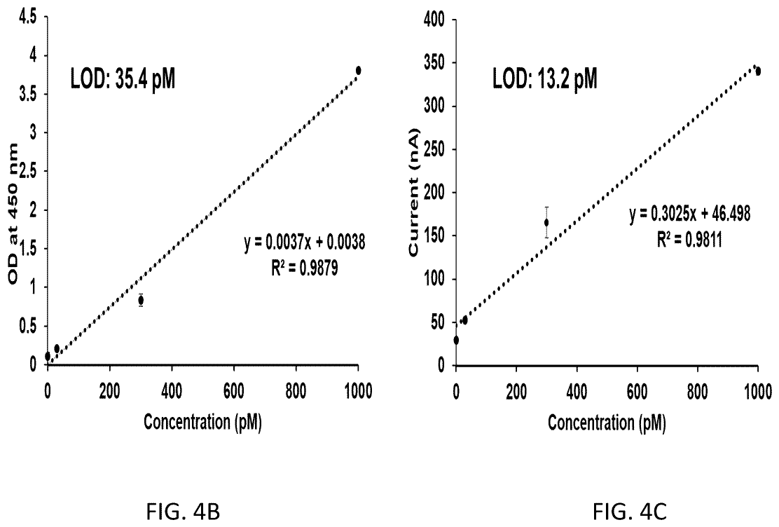

(G) Assay Optimization—Background determination in PBS: To optimize assay development and electrochemical detection, efforts began with the Hcp100 gene, one of the selected gene panels for this study. Wells with 1 nM of Hcp100 target DNA show a significant difference in absorbance as compared to control wells that only contain the capture probe (Cp), or Cp and target (T), or Cp and amplification probes, Ap (no target DNA). FIG. 4A shows that signals obtained from absorbance measurement or optical density (OD) of TMB are strong enough to distinguish from the background signal and that the signal from the amperometry (data not shown) matches up well with the absorbance signal from the TMB for 200 pM of Hcp100 gene target.

(H) Sensitivity testing of Hcp100 genes in PBS: As shown in FIGS. 4B and 4C, a significant amount of signal enhancement was observed in the presence of various target concentrations (1 nM down to 25 pM) as compared to the control experiment. This signal enhancement was validated through colorimetric and UV-Vis measurement at 450 nm (FIG. 4B), and the spectroscopic 3-sigma rule based LOD (limit of detection) was at the pM level. The LOD was calculated, followed by the 3-sigma rule. The equation is LOD=3.3×standard deviation of the regression line (σ)/Slope(S). A 3σ-rule is widely used to determine the signal-to-noise ratio for estimating the detection limit. (See, for example, Rana et al., “Reprogrammable multiplexed detection of circulating oncomiRs using hybridization chain reaction,” Chemical Communications, 52: 3524-27 (2016), which is incorporated herein by reference.) The sensitivity testing confirmed the LOD is within pM ranges and showed that the sensitivity (FIG. 4B, R²=0.9879) is highly reproducible. A similar finding was observed from electrochemical analysis. Additionally, in order to normalize the intra-assay or plate-to-plate variances, the data was normalized with background subtraction. Overall, the standard curve with R²=0.9811 value from electrochemical amperometric peak analysis (FIG. 4C) shows excellent linearity and correlation between peak current and concentration of target DNA.

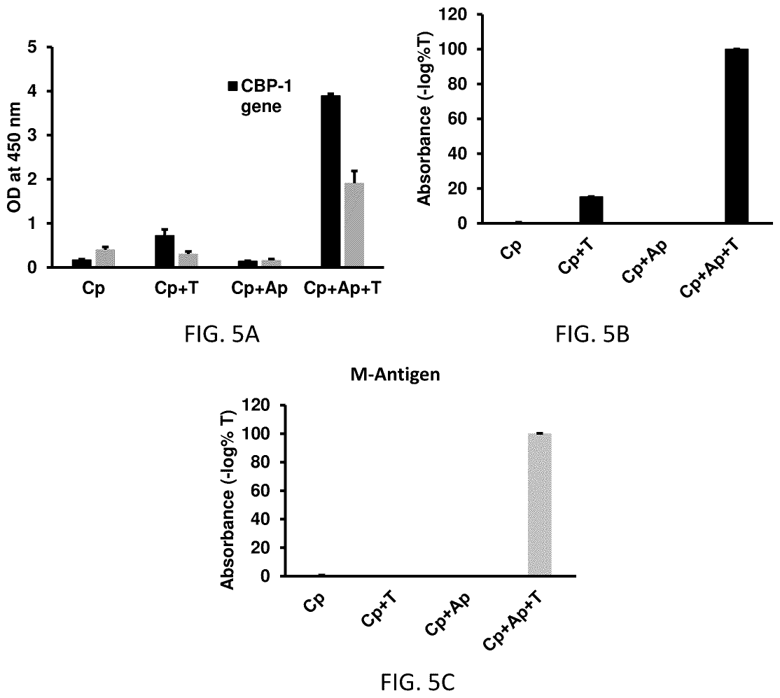

(I) Assay Versatility—In vitro absence test of CBP1 and M antigen genes: As shown in FIGS. 5A through 5C, wells with target DNA show a significant difference in optical density (OD), as compared to control wells that only contain the capture probe (Cp), or Cp and target (T), or Cp and amplification probes, Ap (no target DNA), or Cp+Ap+T. More specifically, FIG. 5A shows that signals obtained from absorbance measurement of TMB are strong enough (Cp+Ap+T) to distinguish from the background signal (Cp or Cp+T or Cp+Ap) and that the signal from the amperometry (data not shown) matches up well with the absorbance signal from the TMB for 1 nM of CBP1 or M antigen gene target. Additionally, in order to normalize intra-assay or plate-to-plate variances, in FIGS. 5B and 5C, the data was normalized with background subtraction and represented it as absorbance (%) for CBP1 (FIG. 5B) and M antigen genes (FIG. 5C). This result re-confirms that the assay's absence test is reproducible, and the functionality of the assay is promising to evaluate the sensitivity and specificity of CBP1 and M antigen genes in biological samples.

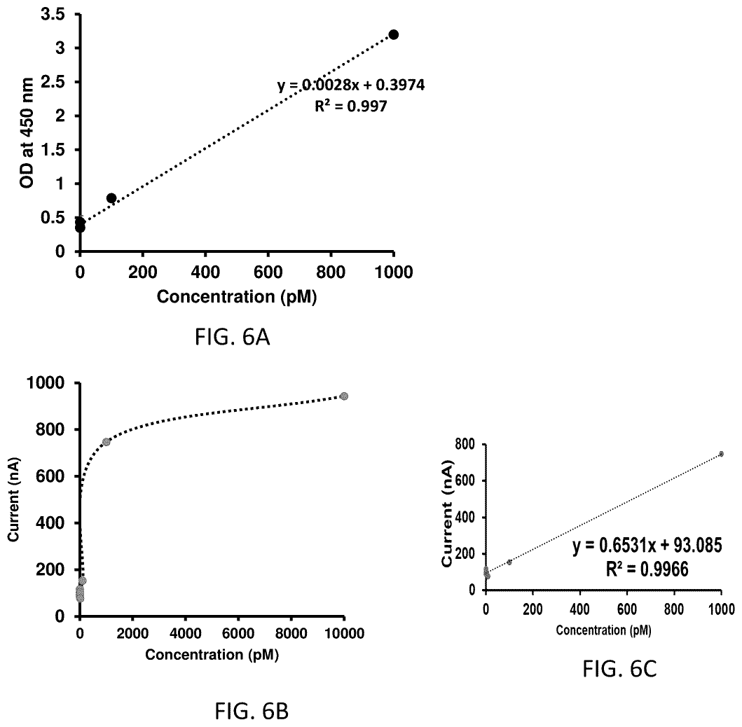

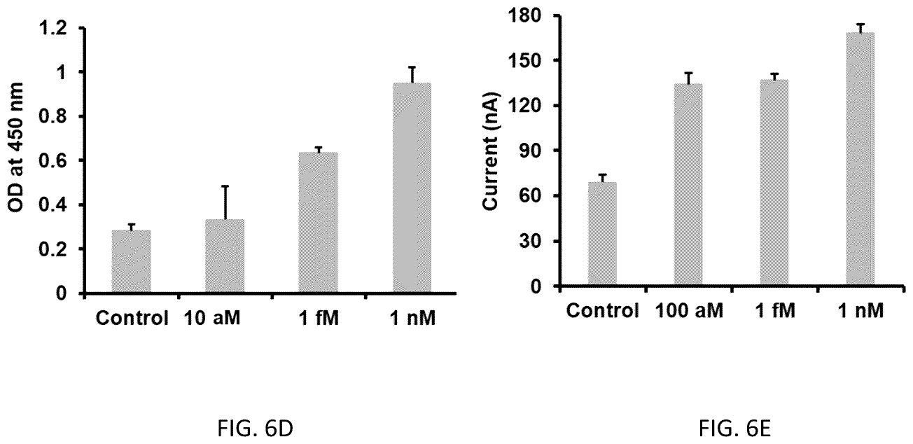

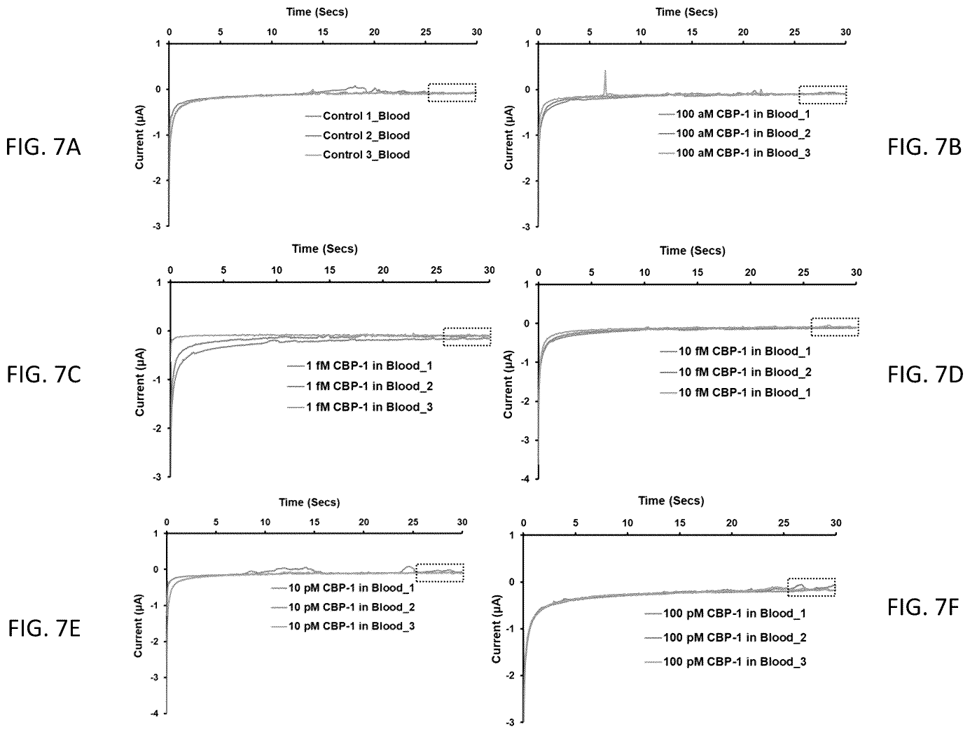

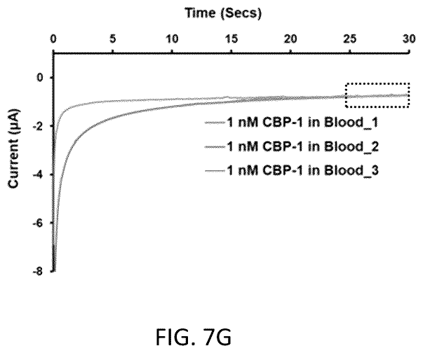

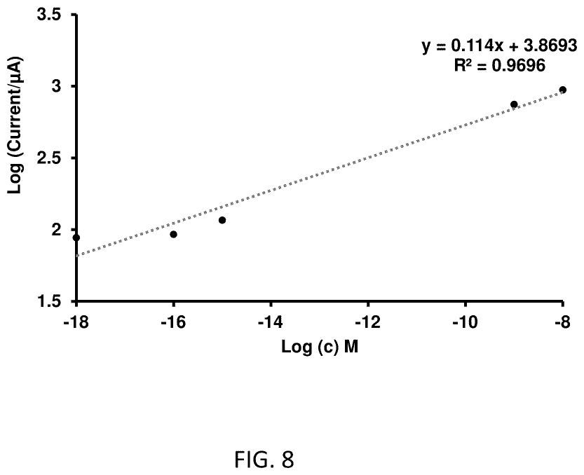

(J) Sensitivity Validation—Sensitivity Evaluation of CBP1 and M antigen in Blood Samples: As shown in FIGS. 6A through 6E, a significant amount of signal enhancement in various target concentration settings (10 nM down to 100 aM) was observed compared to a control experiment for CBP1 and M antigen genes (highly reported genes for Histoplasma compared to the other gene panels) in human whole blood samples. Furthermore, this signal enhancement was validated through UV-Vis measurement at 450 nm with a statistical LOD of 3.84 fM for CBP1 (FIG. 6A, R²=0.997) and a femtomolar (fM) level of electrochemical LOD for CBP1 (FIG. 6C, R²=0.9966) in human whole blood samples. A representative series of electrochemical chronoamperometry measurements of CBP-1 is shown in FIGS. 7A through 7G. The log scale-based low and high ranges of concentrations and their data analysis and LOD are shown in FIG. 8 (R²=0.9696). The log-scale-based statistical LOD is 20.1 aM.

After the initial confirmation of spectroscopic and electrochemical sensitivity of CBP1 gene, the spectroscopic (FIG. 6D) and electrochemical (FIG. 6E) sensitivities for M antigen gene in human whole blood samples were repeated. Again, highly reproducible dynamic ranges of sensitivity were observed. Overall, a standard curve with R²=0.9966 value from electrochemical amperometry peak analysis (FIG. 6C) shows excellent linearity and correlation between peak current and concentration of target DNA.

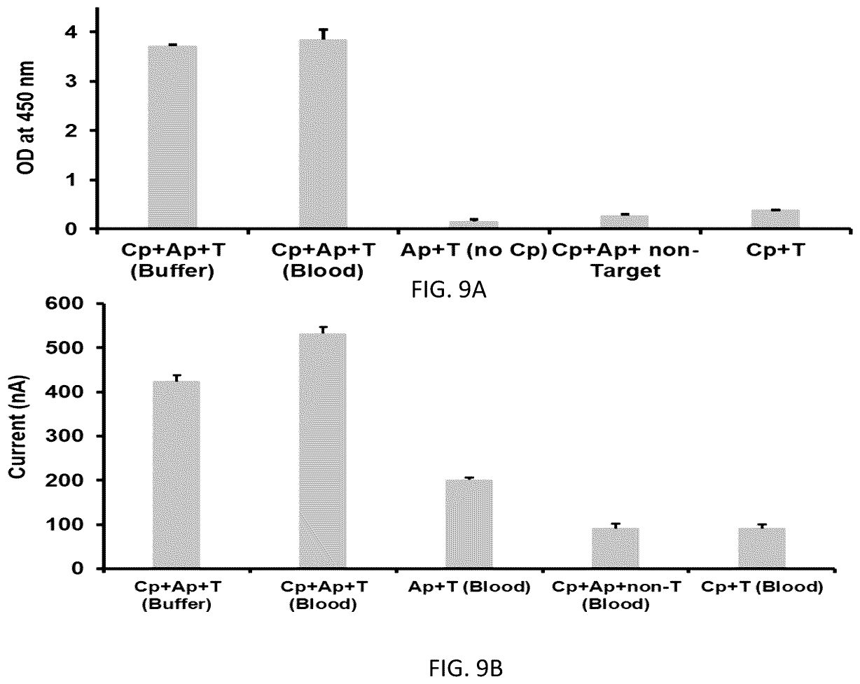

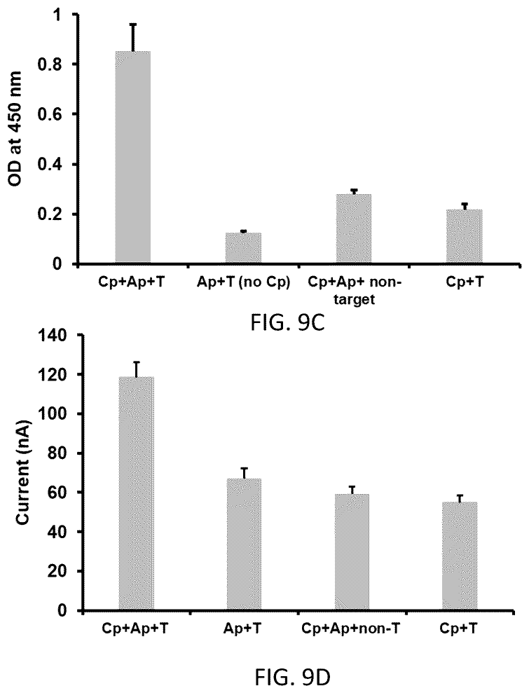

(K) Specificity Validation—Specificity Evaluation of CBP1 and M antigen in Blood Samples: To verify assay specificity, whole blood samples were utilized to detect exogenously enriched CBP1 (FIGS. 9A and 9B) and M antigen (FIGS. 9C and 9D) gene targets. As can be seen, the assay successfully detected 1 nM of CBP1 and M antigen in complex biological matrices. Next, the specificity of CBP1 and M antigen genes were tested in clinical blood samples (FIGS. 9A through 9D). In the absence of target (FIGS. 9A and 9B and FIGS. 9C and 9D), the “sandwich” assay showed a very light blue color that transitioned to a light to dark yellow color in the presence of H₂SO₄. When the target was present, a bright and dark blue color was observed due to poly-HRP triggered oxidation of TMB, which transitioned to orange when acid was added.

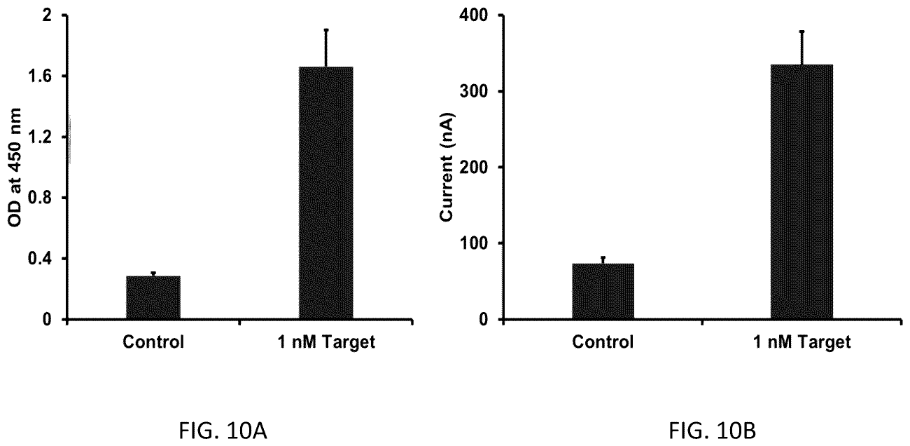

Overall, UV-Vis and electrochemical signals from human whole blood samples shown in FIGS. 9A through 9D indicate that the assay is highly specific and produces less than 2% signal with non-specific target analytes. Similarly, the specificity of the enzyme-linked assay was tested for sensitive detection of the Hcp100 gene target from a complex cocktail mixture of the non-target gene in whole blood samples (FIGS. 10A and 10B).

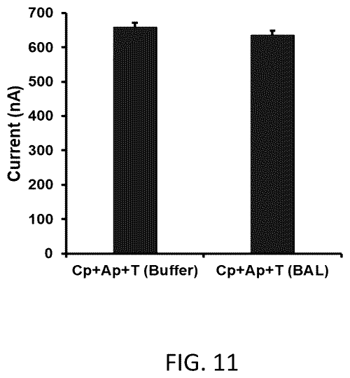

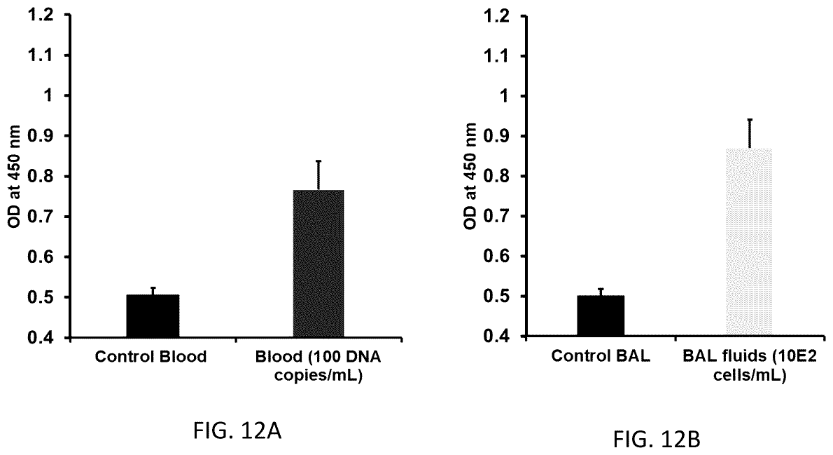

(L) Preclinical utility validation—Testing of complex BAL sample: To verify assay versatility and preclinical utility, the platform was tested with commercial bronchoalveolar lavage (BAL) samples. As seen in FIG. 11, the assay successfully detected 1 nM of the CBP1 gene in a spiked BAL sample. When the target was present, a vivid blue color was observed. Overall, UV-Vis, colorimetric images, and electrochemical signals from the human BAL sample indicated that the assay is highly specific and able to detect the correct target in PBS, blood (previously shown in FIGS. 4A through 4C, FIGS. 5A through 5C, FIGS. 6A through 6E, and FIGS. 9A through 9D), and BAL samples. FIG. 11 confirms assay versatility and preclinical utility in different challenging biological samples. The sensor was also validated with a randomized unbiased sample where the target genes were exogenously enriched in individual biological matrixes and then extracted prior to testing (FIGS. 12A and 12B).

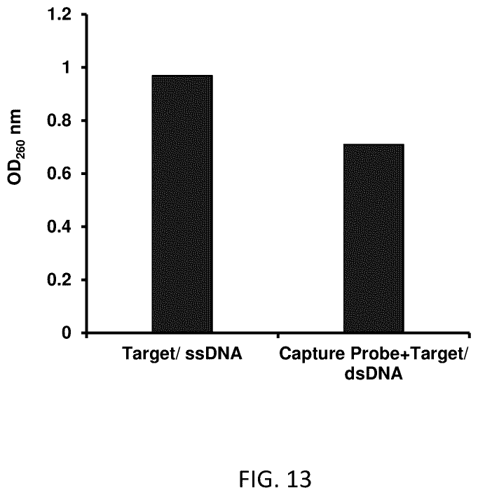

(M) Hybridization efficiency characterization—Photometric characterization of DNA hybridization efficiency: To understand hybridization efficiency, probe-target hybridization was systematically optimized and characterized in different conditions. First, consideration was given to the photometric measurement where single-stranded DNA (Hcp100 gene target) can be differentiated from double-stranded DNA based on their respective absorbance (OD₂₆₀) values. Double-stranded DNA (Hcp100 gene target and its capture probe incubated at 1:1 ratio) absorbs less strongly than denatured/ssDNA due to stacking interactions between the bases. Next, ssDNA probe and dsDNA (probe-target) hybridization were tested using a Nanodrop spectrophotometer (DeNovix DS-11 FX+, DeNovix, Inc., Wilmington, DE). The results shown in FIG. 13 indicate that Hcp100 ssDNA has higher absorbance or OD₂₆₀ value than Hcp100 dsDNA.

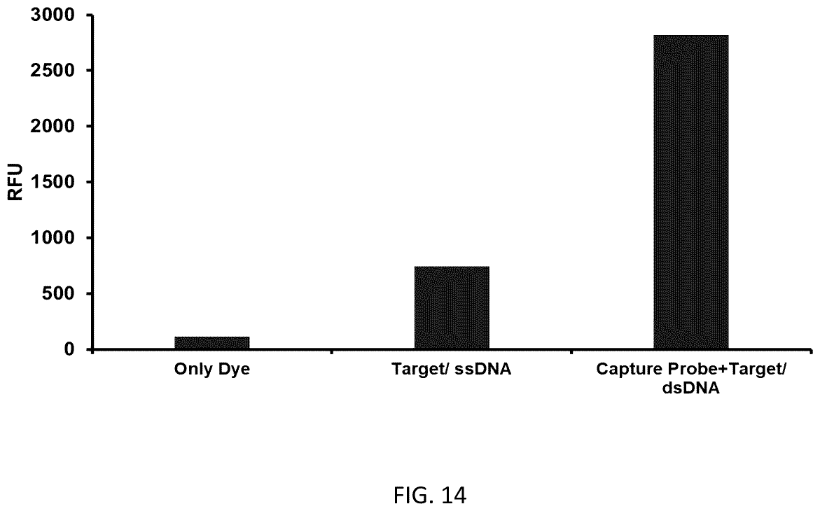

(N) Hybridization efficiency characterization—Fluorogenic characterization of DNA hybridization efficiency: The commercially available double strand-specific DNA intercalation fluorescence dye SYBR Green I (SG I) was used to characterize DNA hybridization efficiency. Prior to utilization, the dye was diluted (1:10,0000×) according to the manufacturing protocol, which specifies that SG I is more specific to dsDNA due to its charge binding affinity to the DNA base. As indicated in FIG. 14, the buffer (referred to as blank or control) with SG I dye did not fluoresce under transilluminator operation. A light orange color fluorescence was observed with ssDNA, which transitioned to green due to intercalation in the presence of dsDNA. Also, although not shown, the ssDNA and dsDNA with dye intercalation interaction could be clearly observed. Subsequently, the fluorescence emission from individual tubes was measured using a Nanodrop based fluorometer, DeNovix DS-11 FX+(SG I excitation wavelength at ˜470 nm and emission wavelength at ˜525 nm). A distinguished fluorescence intensity difference between ssDNA and dsDNA in Hcp100 samples at room temperature after 15 minutes of incubation was observed, which confirms that an efficient hybridization is taking place at room temperature.

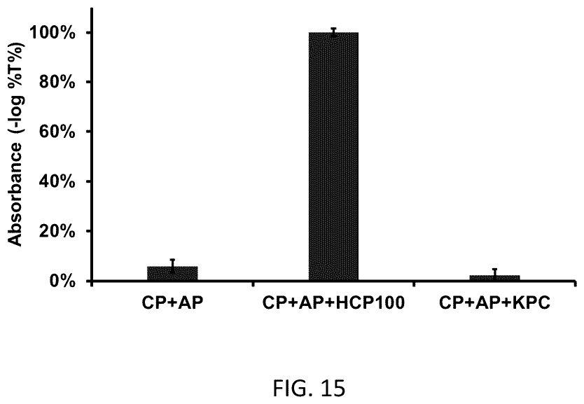

(0) Assay Validation—Specificity testing of Hcp100 genes in PBS: The specificity of the assay for sensitive detection of the Hcp100 gene target from a complex cocktail mixture of KPC (a non-target gene from enteric bacteria) was tested. In the absence of target, the assay showed a very light blue color that transitioned to light to dark yellow color in the presence of H₂SO₄. By contrast, when the target was present, a bright and dark blue color was observed due to poly-HRP triggered oxidation of TMB, which transitioned to bright orange when acid was added. The colorimetric assay required a few seconds to be visualized with the naked eye. Overall, as can be seen in FIG. 15, UV-Vis signals showed that the assay is highly specific to detect the correct target in a cocktail mixture of target and non-target, and it only produces less than 2% signal with non-specific target analytes.

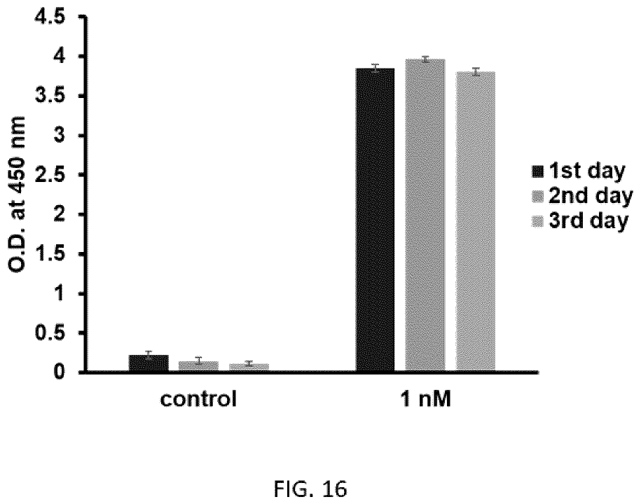

(P) Assay Performance—Reproducibility of Assay in PBS: From FIG. 16, there can be seen the reproducibility of the assay to make sure its performance is stable and reproducible in different time settings. More specifically, as can be seen in FIG. 16, barely any variation of reproducibility in UV-Vis's measurement was observed, and the statistical coefficient variation was only 0.24 for control and 0.01 for 1 nM of target Hcp100 gene, which is acceptable.

(Q) Assay Performance—Electrochemical Hcp100 gene detection in blood samples: In order to verify assay performance in complex biological samples, commercial whole blood samples were utilized to detect exogenously enriched Hcp100 gene target. As seen in FIGS. 10A and 10B, the assay successfully detected 1 nM of Hcp100 gene in spiked commercial whole blood samples. The UV-Vis's data measured at 450 nm are shown in FIG. 10A. The graphical representation of electrochemical measurement is shown in FIG. 10B. This assay was explored and reported with an LOD of 13.2 pM, and with incorporation of poly-HRP and the reduced assay turnaround time (less than 90 mins), and now it shows great potential to yield high reproducibility, sensitivity, specificity as well as potential detection in commercial blood samples. This data indicates that the assay is ready to be tested in blood samples for other targets, such as CBP1 and M antigen gene.

(R) Testing of randomized and unbiased and samples with the Assay: The assay was utilized to re-validate its pre-clinical utility through testing H. capsulatum gDNA or yeast spiked samples (blood and BAL fluids) in which the target genes were exogenously enriched in individual biological matrixes and then extracted prior shipping to the present inventors, followed by a standard operating procedure in a BSL (biosafety level) 3 lab. As seen in FIGS. 12A and 12B, in presence of the target gene CBP1, the assay produced an anticipated higher signal compared to control. A colorimetric based UV-Vis signal at 450 nm confirmed the specific detection of CBP1 with a LOD of 100 DNA copies/mL in blood (FIG. 12A) and 100 (10E2) cells/mL in BAL fluids (FIG. 12B), thereby proving the preclinical utility of the assay in various challenging biological samples.

Some additional features, aspects and/or advantages that may apply to at least some embodiments of the present invention are discussed below.
The treatment of fungal infections is increasingly crucial for human health. Late diagnosis of fungal infections can be life-threatening and compromise the effectiveness of available therapeutic interventions. Accurate and early diagnosis can be a lifesaver to assist in developing therapeutic agents, directing antifungal management, and preventing misuse of antibiotics.
The present application discloses how a practical DNA-based electrochemical sensor can precisely detect a single target gene using sequence-specific biotinylated probes and streptavidin poly-HRP80 conjugates.
The strategic involvement of poly-HRP80 conjugates within the probe-target hybridization provides highly sensitive detection of DNA levels (as low as 100 aM) in blood and BAL samples.
The challenges of accurate detection of fungal genes can be addressed using a biosensor assay that involves the integration of poly-HRP80 conjugates enzyme and sequence-specific capture probe (Cp) and labeling/amplification probes (P1, P2, and P3) followed by the TMB-H₂O₂ reaction.
The preclinical utility of the DNA sensor platform disclosed herein is not restricted to detect only fungal genes. The versatility of this technology allows for target-specific probe design to detect other pathogen genes and high throughput performance is enabled by the use of standard streptavidin 96-well plates. Using the same platform, the detection of other clinically relevant infectious genes and disease biomarkers with different probes may be effected.
The present invention provides a practical point-of-care (POC) test for rapid and accurate detection of Histoplasma genomic sequences in the blood (for systemic infections), sputum, and bronchoalveolar lavage fluid (BAL, for respiratory infections) to address unmet diagnostics need for histoplasmosis.
The present invention provides ultra-sensitive detection of DNA t olecules with limit of detection (LOD) values down to 100 attomolar (aM), sufficient to meet the clinical diagnostic need. In addition, the sample to result turnaround time is relatively fast (≤90 mins).
The present invention provides an electrochemical assay capable of rapid, cost-effective, highly specific, and sensitive detection of Histoplasma specific gene for diagnosis of histoplasmosis in whole blood, cerebrospinal fluid (CSF), and bronchoalveolar lavage (BAL) samples.
The present invention may be used to detect drug-resistant organisms in the gut microbiome in less than 90 minutes, with a LOD of 100 attomolar (aM) DNA (˜10 colony-forming units per mL).
The present invention obviates the need for techniques that involve polymerase chain reaction (PCR), which is both time-intensive and labor-intensive.

The embodiments of the present invention described above are intended to be merely exemplary and those skilled in the art shall be able to make numerous variations and modifications to it without departing from the spirit of the present invention. All such variations and modifications are intended to be within the scope of the present invention.

What is claimed is:

1. A method of detecting a nucleic acid of interest, the method comprising the steps of:
(a) providing a surface;
(b) securing a capture probe to the surface, wherein the capture probe comprises a capture probe nucleic acid, wherein the capture probe nucleic acid is designed to hybridize with specificity to a first portion of the nucleic acid of interest;
(c) then, exposing the capture probe to a sample, wherein, if the sample comprises the nucleic acid of interest, the first portion of the nucleic acid of interest hybridizes to the capture probe nucleic acid of the capture probe;
(d) then, adding a first labeling probe, wherein the first labeling probe comprises a first labeling nucleic acid, wherein the first labeling nucleic acid is designed to hybridize with specificity to a second portion of the nucleic acid of interest, wherein, if the nucleic acid of interest is captured, the first labeling probe hybridizes to the second portion of the captured nucleic acid of interest;
(e) then, adding an enzyme label, wherein the enzyme label is designed to bind to the first labeling probe, wherein, if the first labeling probe is hybridized to the captured nucleic acid of interest, the enzyme label becomes coupled to the captured nucleic acid of interest;
(f) then, adding a substrate whose reaction is catalyzed by the enzyme label, wherein, if the enzyme label is coupled to the captured nucleic acid of interest, the substrate reacts; and
(g) then, determining, photonically and/or electrochemically, if any substrate reacts in step (f).

2. The method as claimed in claim 1 wherein the nucleic acid of interest is at least a portion of a fungal gene.

3. The method as claimed in claim 2 wherein the fungal gene is a gene of Histoplasma capsulatum.

4. The method as claimed in claim 3 wherein the fungal gene is selected from the group consisting of the Hcp100 gene, the CBP1 gene, and the M antigen gene.

5. The method as claimed in claim 3 wherein the fungal gene is the Hcp100 gene.

6. The method as claimed in claim 1 wherein the surface is a well of a multiwell plate.

7. The method as claimed in claim 1 wherein one of the well and the capture probe is biotinylated and the other of the well and the capture probe is modified with streptavidin.

8. The method as claimed in claim 7 wherein the capture probe is biotinylated and the well is coated with streptavidin.

9. The method as claimed in claim 6 wherein the multiwell plate includes an electrode.

10. The method as claimed in claim 1 wherein the sample is one of a whole blood sample, a blood plasma sample, a sputum sample, a bronchoalveolar lavage sample, and a sample derived from one or more thereof.

11. The method as claimed in claim 1 wherein one of the first labeling probe and the enzyme label is biotinylated and the other of the first labeling probe and the enzyme label is modified with streptavidin.

12. The method as claimed in claim 11 wherein the first labeling probe is biotinylated and the enzyme label is modified with streptavidin.

13. The method as claimed in claim 1 further comprising, after step (c) and before step (e), adding two or more additional labeling probes, the two or more additional labeling probes comprising a second labeling probe and a third labeling probe, wherein the second labeling probe comprises a second labeling nucleic acid and the third labeling probe comprises a third labeling nucleic acid, wherein the second labeling nucleic acid is designed to hybridize with specificity to a third portion of the nucleic acid of interest and the third labeling nucleic acid is designed to hybridize with specificity to a fourth portion of the nucleic acid of interest, wherein the enzyme label is also designed to bind to the second labeling probe and the third labeling probe, and wherein, if the nucleic acid of interest is captured, the second labeling probe hybridizes to the third portion of the captured nucleic acid of interest and the third labeling probe hybridizes to the fourth portion of the captured nucleic acid of interest.

14. The method as claimed in claim 13 wherein the enzyme label is a streptavidin-modified horseradish peroxidase conjugate.

15. The method as claimed in claim 14 wherein the streptavidin-modified horseradish peroxidase conjugate includes five identical horseradish peroxidase homopolymer blocks, each horseradish peroxidase homopolymer block comprising 80 horseradish peroxidase monomers.

16. The method as claimed in claim 14 wherein the substrate comprises 3,3′,5,5′-tetramethylbenzidine and wherein the substrate is accompanied by hydrogen peroxide.

17. The method as claimed in claim 1 further comprising, after step (f) and before step (g), stopping the reaction of the substrate by the addition of an acid.

18. The method as claimed in claim 1 wherein photonically determining if any substrate reacts in step (f) comprises detecting any colorimetric change of the substrate and comparing said colorimetric change, if any, to standards and negative or positive control samples.

19. The method as claimed in claim 1 wherein photonically determining if any substrate reacts in step (f) comprises obtaining an absorbance reading of the substrate and comparing said absorbance reading to standards to determine the amount of any reacted substrate.

20. The method as claimed in claim 19 wherein the surface is a well of a multiwell plate and wherein the absorbance reading is taken of any contents of the well while said contents are still in the well.

21. The method as claimed in claim 1 wherein electrochemically determining if any substrate reacts in step (f) comprises performing chronoamperometric analysis of any reacted substrate and comparing said chronoamperometric analysis to standards to determine the amount of any reacted substrate.

22. The method as claimed in claim 21 wherein the surface is a well of a multiwell plate, the well including an electrode, and wherein the chronoamperometric analysis is performed on any contents of the well while said contents are still in the well.

23. The method as claimed in claim 1 wherein the surface is a well, the method further comprising, after step (c) and before (d), after step (d) and before step (e), and after step (e) and before step (f), removing any non-specific binding from the well.

24. A system for use in detecting a target nucleic acid, the system comprising:
(a) a multiwell plate, the multiwell plate comprising a plurality of wells;
(b) a plurality of containers, wherein the plurality of containers comprises
(i) a first container, the first container comprising a capture probe solution, the capture probe solution comprising a capture probe nucleic acid, wherein the capture probe nucleic acid is designed to bind to a well of the multiwell plate and to hybridize with specificity to a first portion of the target nucleic acid,
(ii) a second container, the second container comprising a labeling probe solution, the labeling probe solution comprising a plurality of labeling nucleic acids, each of the plurality of labeling nucleic acids being designed to hybridize with specificity to portions of the target nucleic acid that are different from one another and different from the first portion of the target nucleic acid,
(iii) a third container, the third container comprising an enzyme label solution, the enzyme label solution comprising an enzyme label, the enzyme label being designed to bind to the labeling nucleic acids,
(iv) a fourth container, the fourth container comprising a substrate solution, the substrate solution comprising a substrate that reacts in the presence of the enzyme label,
(c) one or more photonic and/or electrochemical measuring devices, the one or more photonic and/or electrochemical measuring devices being designed to measure the amount of any reacted substrate in the well;
(d) a compute device, coupled to the one or more photonic and/or electrochemical measuring devices, for comparing the measured amount of any reacted substrate in the well to standards to determine the amount of the target nucleic acid in the well; and
(e) an output device, coupled to the compute device, for displaying the amount of the target nucleic acid in the well.

25. The system as claimed in claim 24 wherein at least at least a portion of one well of the multiwell plate is coated with streptavidin.

26. The system as claimed in claim 25 wherein at least one well of the multiwell plate further comprises an electrode.

27. The system as claimed in claim 24 wherein the target nucleic acid is GAGATCTAGTCGCGGCCAGGTTCACGGAGGACAACGAGTGGTACCGCGCAA AAATACGGAGAAACGACCGTGAAGCGAAAAAAGCCGACGTCGTTTACATCG ACTACGGCAACTCCGAAA CCGTTCCGTGGAC (SEQ ID NO: 1), wherein the capture probe nucleic acid is /5Biosg/TG AAC CTG GCC GCG ACT AGA TCT C (SEQ ID NO: 2), and wherein the labeling nucleic acids are TTG CGC GGT ACC ACT CGT /3Bio/ (SEQ ID NO: 3), CGA TGT AAA CGA CGT CGG CT/3Bio/ (SEQ ID NO: 4), and GTC CAC GGA ACG GTT TCG /3Bio/ (SEQ ID NO: 5).

28. The system as claimed in claim 24 wherein the target nucleic acid is TAT AAA TAT CAG CTC CTT CAC ACT CAG GAA TGG ATG TCT TAC CCT CAA CAT ACA ATC AGC AAG AGA AAA CCC AGC GAA AAT CAC CTC CTC AAT CAA ACA TTC AAA AAA TCT ACG TTC TTT TCC AGA ACA ACC ACT TCG TCA TTC AAA ATG CTT TTC TCC AAG GTT ATC GCT CCT GCT TTC A (SEQ ID NO: 6), wherein the capture probe nucleic acid is /5Biosg/ GTG TGA AGG AGC TGA TAT TTA TA (SEQ ID NO: 7), and wherein the labeling nucleic acids are CGC TGG GTT TTC TCT TGC TG /3Bio/ (SEQ ID NO: 8), CGA AGT GGT TGT TCT GGA AA /3Bio/(SEQ ID NO: 9), and AAA GCA GGA GCG ATA ACC TTG GAG /3Bio/ (SEQ ID NO:

10. .

29. The system as claimed in claim 24 wherein the target nucleic acid is GCC ATA AGG ACG TCA CGA AGG GCT TCA TTG CTA CCG TCA CCG ACA GCG CCG ACG GGC TTT CCA TAC GCG TAT GCA TCC GTA ATA ATC CTG AGC GGG CGA CCT CTT GGG TAT TGC GTT GAG GCG CTC GTG AGC AGG CCG CCG ACG ACG ATC ACG GCA TCG AAG ATC GAG CCG TCG GCG CCG GAA TAG GTC ATG TTC ACG C (SEQ ID NO: 11), wherein the capture probe nucleic acid is /5Biosg/ GAA GCC CTT CGT GAC GTC CTT ATG (SEQ ID NO: 12), and wherein the labeling nucleic acids are TCA GGA TTA TTA CGG ATG CA /3Bio/ (SEQ ID NO: 13), TTC GAT GCC GTG ATC GTC GT /3Bio/ (SEQ ID NO: 14), and CGT GAA CAT GAC CTA TTC /3Bio/ (SEQ ID NO: 15).


SEQUENCE LISTING (15 sequences)

SEQ ID NO: 1 - DNA, 133 bp, synthetic construct
  source 1..133: mol_type = other DNA
gagatctagtcgcggccaggttcacggaggacaacgagtggtaccgcgcaaaaatacggagaaacgaccgtgaagcgaaaaaagccgacgtcgtttacatcgactacggcaactccgaaaccgttccgtggac

SEQ ID NO: 2 - DNA, 24 bp, synthetic construct
  source 1..24: mol_type = other DNA
tgaacctggccgcgactagatctc

SEQ ID NO: 3 - DNA, 18 bp, synthetic construct
  source 1..18: mol_type = other DNA
ttgcgcggtaccactcgt

SEQ ID NO: 4 - DNA, 20 bp, synthetic construct
  source 1..20: mol_type = other DNA
cgatgtaaacgacgtcggct

SEQ ID NO: 5 - DNA, 18 bp, synthetic construct
  source 1..18: mol_type = other DNA
gtccacggaacggtttcg

SEQ ID NO: 6 - DNA, 181 bp, synthetic construct
  source 1..181: mol_type = other DNA
tataaatatcagctccttcacactcaggaatggatgtcttaccctcaacatacaatcagcaagagaaaacccagcgaaaatcacctcctcaatcaaacattcaaaaaatctacgttcttttccagaacaaccacttcgtcattcaaaatgcttttctccaaggttatcgctcctgctttca

SEQ ID NO: 7 - DNA, 23 bp, synthetic construct
  source 1..23: mol_type = other DNA
gtgtgaaggagctgatatttata

SEQ ID NO: 8 - DNA, 20 bp, synthetic construct
  source 1..20: mol_type = other DNA
cgctgggttttctcttgctg

SEQ ID NO: 9 - DNA, 20 bp, synthetic construct
  source 1..20: mol_type = other DNA
cgaagtggttgttctggaaa

SEQ ID NO: 10 - DNA, 24 bp, synthetic construct
  source 1..24: mol_type = other DNA
aaagcaggagcgataaccttggag

SEQ ID NO: 11 - DNA, 199 bp, synthetic construct
  source 1..199: mol_type = other DNA
gccataaggacgtcacgaagggcttcattgctaccgtcaccgacagcgccgacgggctttccatacgcgtatgcatccgtaataatcctgagcgggcgacctcttgggtattgcgttgaggcgctcgtgagcaggccgccgacgacgatcacggcatcgaagatcgagccgtcggcgccggaataggtcatgttcacgc

SEQ ID NO: 12 - DNA, 24 bp, synthetic construct
  source 1..24: mol_type = other DNA
gaagcccttcgtgacgtccttatg

SEQ ID NO: 13 - DNA, 20 bp, synthetic construct
  source 1..20: mol_type = other DNA
tcaggattattacggatgca

SEQ ID NO: 14 - DNA, 20 bp, synthetic construct
  source 1..20: mol_type = other DNA
ttcgatgccgtgatcgtcgt

SEQ ID NO: 15 - DNA, 18 bp, synthetic construct
  source 1..18: mol_type = other DNA
cgtgaacatgacctattc